(19) United States
(12) Patent Application Publication
(10) Pub. No.: US 2025/0237662 A1
(43) Pub. Date: Jul. 24, 2025

(54) RABBIT ANTIBODIES TO HUMAN IMMUNOGLOBULINS G

(71) Applicant: Genzyme Corporation, Cambridge, MA (US)

(72) Inventor: Ruiyin CHU, Westborough, MA (US)

(73) Assignee: Genzyme Corporation, Cambridge, MA (US)

(21) Appl. No.: 18/991,856

(22) Filed: Dec. 23, 2024

Related U.S. Application Data
(63) Continuation-in-part of application No. 17/335,291, filed on Jun. 1, 2021, now Pat. No. 12,215,171.
(60) Provisional application No. 63/033,073, filed on Jun. 1, 2020.

(51) Int. Cl. G01N 33/68; C07K 16/42; G01N 33/533

(52) U.S. Cl. CPC G01N 33/6854; C07K 16/42; G01N 33/533; C07K 2317/24; C07K 2317/33; C07K 2317/92; C07K 2319/60

(57) ABSTRACT

This disclosure anti-human IgG antibodies and antigen-binding portions thereof derived from rabbits and methods of using these antibodies and portions.

### CROSS-REFERENCE TO RELATED APPLICATIONS

This application is a continuation of U.S. patent application Ser. No. 17/335,291, filed Jun. 1, 2021, which claims priority from U.S. Provisional Patent Application No. 63/033,073, filed Jun. 1, 2020. The disclosure of each of the aforementioned priority applications is incorporated herein by reference in its entirety.

### SEQUENCE LISTING

The instant application contains a Sequence Listing that has been submitted electronically in .XML format and is hereby incorporated by reference in its entirety. Said .XML copy, named 022548_C1063_SL.xml, is 114,183 bytes in size and was created on Dec. 20, 2024.

### BACKGROUND OF THE INVENTION

Therapeutic monoclonal antibodies (mAbs) have become one of the fastest growing classes of drugs in recent years and are approved for the treatment of a wide range of indications, from cancer to autoimmune diseases. Preclinical pharmacokinetic characterization of these therapeutic monoclonal antibodies often has to be performed in non-human primates in order to prove efficacy and safety before the initiation of clinical studies. The cynomolgus monkey is a preferred non-human primate for such preclinical studies because it often provides a sufficient level of cross-reactivity with the target of the therapeutic antibody (Iwasaki et al., Drug Metab Pharmacokinet. (2019) 34:55-63). However, the immunoglobulins of the cynomolgus monkey also show a high sequence homology with the immunoglobulins of humans. The lack of high-quality reagent antibodies that can distinguish a human therapeutic molecule from cynomolgus monkey immunoglobulin in the serum constitutes a significant challenge for the bioanalytical measurement of human therapeutic antibodies in non-human primate serum samples because of the high level of protein sequence homology for IgG (Stubenrauch et al., J Pharm Biomed Anal. (2009) 49:1003-8).

Currently, evaluation of therapeutic antibody pharmacokinetics (PK) and pharmacodynamics (PD) in preclinical studies relies on drug specific anti-idiotype antibodies, which are laborious and time-consuming to develop. Each drug candidate would require its own anti-idiotype antibody. There are few options for a universal reagent that can detect all human IgG-based therapeutic antibodies in preclinical studies. There is therefore a need to develop monoclonal antibodies that are universally specific for human IgG but do not bind monkey IgG, in order to accurately measure the levels of therapeutic human IgG-derived mAbs in non-human primates during preclinical studies.

### SUMMARY OF THE INVENTION

The present disclosure provides isolated monoclonal antibodies or antigen-binding portions thereof that bind specifically to human IgG, wherein the antibodies or portions comprise heavy chain complementarity-determining region (CDR) 1-3 and light chain CDR1-3 respectively comprising SEQ ID NOs: 27-32, SEQ ID NOs: 33-38, SEQ ID NOs: 39-44, SEQ ID NOs: 45-50, SEQ ID NOs: 51-56, or SEQ ID NOs: 57-62. The antibodies may be rabbit antibodies or modified from such molecules (including, for example, chimeric antibodies with Fc domains from a non-rabbit species such as mouse, rat, or human).

In some embodiments, the present antibody or portion comprises heavy chain variable domain (VH) and light chain variable domain (VL) respectively comprising SEQ ID NOs: 13 and 19, SEQ ID NOs: 14 and 20, SEQ ID NOs: 15 and 21, SEQ ID NOs: 16 and 22, SEQ ID NOs: 17 and 23, or SEQ ID NOs: 18 and 24. The antibodies may be rabbit antibodies or modified from such molecules.

In some embodiments, the present antibody comprises a heavy chain constant region amino acid sequence of SEQ ID NO: 25, and/or a light chain constant region amino sequence of SEQ ID NO: 26. In further embodiments, the antibody comprises a heavy chain and a light chain having the amino acid sequences of SEQ ID NOs: 63 and 69, SEQ ID NOs: 64 and 70, SEQ ID NOs: 65 and 71, SEQ ID NOs: 66 and 72, SEQ ID NOs: 67 and 73, or SEQ ID NOs: 68 and 74, respectively, with or without the leader sequences.

In certain embodiments, the present antibody or antigen-binding portion comprises a detectable label.

The present disclosure also provides a composition or a kit comprising the present monoclonal antibody or antigen-binding portion in an aqueous buffered solution.

In other aspects, the present disclosure provides an isolated nucleic acid molecule encoding the heavy chain, the light chain, or both, of the present monoclonal antibody or antigen-binding portion. In some embodiments, the nucleic acid molecule comprises SEQ ID NOs: 1, 2, 3, 4, 5, 6, 7, 8, 9, 10, 11, and/or 12. In further embodiments, the nucleic acid molecule comprises SEQ ID NOs: 1 and 7, SEQ ID NOS: 2 and 8, SEQ ID NOs: 3 and 9, SEQ ID NOs: 4 and 10, SEQ ID NOs: 5 and 11, or SEQ ID NOs: 6 and 12. Also provided herein is an expression construct comprising the nucleic acid molecule, and a host cell (e.g., a mammalian cell) comprising nucleotide sequences encoding the heavy chain and the light chain of the present monoclonal antibody or antigen-binding portion. The present disclosure also provides a method of producing an antibody or an antigen-binding portion thereof, comprising culturing the host cell under conditions that allow expression of the heavy chain and light chain of the antibody or portion, and isolating the antibody or portion from the cultured cell or the supernatant of the cell culture.

In another aspect, the present disclosure provides a method of detecting human IgG or a fragment thereof in a sample, comprising contacting the sample with one or more monoclonal antibodies or antigen-binding portions described herein. The sample (e.g., a tissue sample such as a blood, serum, or plasma sample, or a biopsy sample) may be obtained from, for example, an animal that has been administered with an antibody comprising a human IgG constant region (e.g., human IgG1, IgG2, IgG3, or IgG4 constant region) or a fragment thereof (e.g., a Fab or F(ab′)2 fragment). The animal may be, for example, a non-human primate such as cynomolgus monkey or rhesus monkey.

Other features, objectives, and advantages of the invention are apparent in the detailed description that follows. It should be understood, however, that the detailed description, while indicating embodiments and aspects of the invention, is given by way of illustration only, not limitation. Various changes and modifications within the scope of the invention will become apparent to those skilled in the art from the detailed description.

### BRIEF DESCRIPTION OF THE DRAWINGS

FIG. 1 is a panel of Biacore sensorgram graphs showing the binding of six rabbit recombinant antibody clones to whole human IgG1 (hIgG1), whole human IgG4 (hIgG4), human Fab (hFab), and cynomolgus monkey IgG (cynoIgG).

FIG. 2A and FIG. 2B show the alignments of the heavy (FIG. 2A) and light (FIG. 2B) chain amino acid sequences, respectively, of the six rabbit recombinant antibody clones using Clustal Omega and the Kabat numbering system. CDRs (see also Zhang and Ho, MABS (2017) 9 (3): 419-29) are underlined and in in boldface. The leader sequences, the start of the variable domains, and the start of the constant regions are marked as shown.

FIG. 2C shows the phylogenetic trees of the VH and VL sequences of the six rabbit antibody clones.

FIGS. 3A and 3B show the epitope determination of the six rabbit anti-hIgG clones by Biacore competition assay (FIG. 3A) and Biacore kinetics assay sensorgram (FIG. 3B). Antibodies MCA5748G (Bio-Rad), 19B1, 11F9 and mouse anti-hIgG mAb (Southern Biotech Cat #9042-01) were first biotinylated and captured on Biacore SA chip at 500-600 RUs, and 8 times 1:2 serially diluted (80-0 nM) hIgG1 antibody (isatuximab; “isa”) was premixed with each of the competition antibodies (240 nM). When the pre-mixed competition antibody binds to hIgG1 at the same site as the antibody captured on the chip surface, it prevents hIgG1 from binding to the captured antibody.

FIGS. 4A-4C show a Gyrolab™ immunoassay using clone 16F5 as the capture reagent, and an overlay graph of hIgG4 in the assay matrixes containing 0%, 4%, 10% and 25% cynomolgus monkey serum (FIG. 4A); a standard curve graph of hIgG4 in the assay matrix containing 4% cynomolgus monkey serum (FIG. 4B); and quality controls in the same assay matrix containing 4% cynomolgus monkey serum (FIG. 4C). Clone 16F5 was biotinylated and used as the capture reagent. The human IgG4 antibody (hIgG4) served as the assay standard was diluted 1 to 4 at the range of 1200-0.30 ng/ml. Alexa Fluor647-conjugated Goat anti-human IgG was used for detection.

FIGS. 5A and 5B show a comparison of different clones used as capture reagents for detection of hIgG4 and hFab. FIG. 5A shows a standard curve of all six rabbit anti-hIgG mAb clones, plus Gyrolab™ and MCA5748G capture agents, for the detection of hIgG4. FIG. 5B shows the same set of capture reagents used for the detection of hIgG Fab. Note that the hFab cannot be detected by the Gyrolab™ capture agent or by the MCA5748G capture agent.

FIG. 6A shows a comparison of four antibodies used in a precipitation and acid dissociation (PandA) assay. The dynamic range and sensitivity of two rabbit anti-human IgG clones, 2C5 and 11G5, were compared with a commercial mouse anti-Fc mAb, clone JDC-10 (Southern Biotech, Cat. No. 9040-01). pAb: a rabbit polyclonal anti-drug antibody (drug: a monoclonal antibody with a human IgG4 constant region). ECL: electrochemiluminescence.

FIGS. 6B and 6C show the performance of clones 2C5 and 11G5, respectively, in human, rat, monkey, and mouse serum matrices in a PandA assay. Pos ctrl: positive control.

### DETAILED DESCRIPTION OF THE INVENTION

The present disclosure provides rabbit monoclonal antibodies that bind to human immunoglobulins G and Fab fragments thereof with high affinity and yet do not bind at a detectable level to IgG of non-human primates such as monkey (e.g., cynomolgus monkey or rhesus monkey). These rabbit antibodies are particular useful as reagents for detecting human IgG or fragments thereof in preclinical pharmacology studies of therapeutic human IgG-based antibodies in cynomolgus monkey or another non-human primate. For example, these rabbit antibodies can be used to study therapeutic antibodies that are fully human IgG antibodies, humanized IgG antibodies, chimeric antibodies having human IgG constant regions, and Fab fragments thereof. The present rabbit antibodies also can be used in preclinical immunohistochemistry studies and in manufacturing process development for human IgG-based therapeutic antibodies (e.g., whole antibodies including mono-specific, bi-specific, and tri-specific antibodies, as well as Fab fragments thereof).

The present rabbit monoclonal antibodies bind to three distinct epitopes on human IgG, which epitopes are further distinct from that bound by the commercially available mouse anti-hIgG antibody MCA5748G. Rabbit monoclonal antibodies offer several advantages over traditional mouse monoclonal antibodies. These advantages include higher binding affinity and specificity and more diverse epitope recognition. The rabbit's immune system is evolutionarily distinct from that of a rodent and it uses different mechanisms to generate, diversify, and optimize the affinity of the antibodies it produces. Additionally, the rabbit's immune system can recognize smaller-sized epitopes that are not immunogenic in mice, while maintaining the ability to produce strong immune responses. Therefore, the rabbit antibodies described herein are advantageous over mouse antibodies.

### Rabbit Anti-hIgG Antibodies

The present disclosure provides antibodies that bind specifically (i.e., with high affinity) to human IgGs and antigen-binding portions thereof (e.g., Fab and F(ab′)₂). These antibodies do not bind at a detectable level to immunoglobulins (such as IgGs) from other species commonly used in preclinical studies (e.g., such as mice, rats, rabbits, non-human primates, or dogs).

As used herein, the term “affinity” refers to a measure of the attraction between an antigen and an antibody. The intrinsic attractiveness of the antibody for the antigen is typically expressed as the binding affinity equilibrium constant (K_{D}) of a particular antibody-antigen interaction. An antibody is said to specifically bind to an antigen when the binding affinity is high, i.e., with a K_{D} of ≤100 nM (e.g., ≤10 nM or ≤1 nM). A K_{D} binding affinity constant can be measured, e.g., by surface plasmon resonance (Biacore®), for example using the Biacore® T200 from Biacore. The binding affinity of a particular antibody-antigen interaction can also be shown by a standard concentration-response curve, for example using a Gyrolab™ xPlore from Gyros Protein Technologies. In some embodiments, the present antibodies bind to human IgG and Fab fragments derived therefrom with a K_{D} no greater than 2 nM while showing no detectable binding to cynomolgus monkey IgG in a Biacore assay.

The antibodies exemplified herein bind to three distinct epitopes on hIgG and Fab fragments thereof. As used herein, the term “epitope” refers to a portion (determinant) of an antigen that specifically binds to an antibody or a related molecule such as a bi-specific binding molecule. Epitopic determinants generally consist of chemically active surface groupings of molecules such as amino acids or carbohydrate or sugar side chains and generally have specific three-dimensional structural characteristics, as well as specific charge characteristics. An epitope may be “linear” or “conformational.” In a linear epitope, all of the points of interaction between a protein (e.g., an antigen) and an interacting molecule (such as an antibody) occur linearly along the primary amino acid sequence of the protein. In a conformational epitope, the points of interaction occur across amino acid residues on the protein that are separated from one another in the primary amino acid sequence. Once a desired epitope on an antigen is determined, it is possible to generate antibodies to that epitope using techniques well known in the art. For example, an antibody to a linear epitope may be generated, e.g., by immunizing an animal with a peptide having the amino acid residues of the linear epitope. An antibody to a conformational epitope may be generated, e.g., by immunizing an animal with a mini-domain containing the relevant amino acid residues of the conformational epitope. An antibody to a particular epitope can also be generated, e.g., by immunizing an animal with the target molecule of interest (e.g., IgG or Fab) or a relevant portion thereof, then screening for binding to the epitope.

One can determine whether an antibody binds to the same epitope or competes for binding with an anti-hIgG antibody of the present disclosure by using methods known in the art, including, without limitation, competition assays, epitope binning, and alanine scanning. In some embodiments, one allows the anti-hIgG antibody of the present disclosure to bind to hIgG under saturating conditions, and then measures the ability of the test antibody to bind to hIgG. If the test antibody is able to bind to hIgG at the same time as the reference anti-IgG antibody, then the test antibody binds to a different epitope than the reference anti-IgG antibody. However, if the test antibody is not able to bind to hIgG at the same time, then the test antibody binds to the same epitope, an overlapping epitope, or an epitope that is in close proximity to the epitope bound by the anti-IgG antibody of the present disclosure. This experiment can be performed using, e.g., ELISA, RIA, BIACORE™, SPR, Bio-Layer Interferometry or flow cytometry. To test whether an anti-hIgG antibody cross-competes with another anti-IgG antibody, one may use the competition method described above in two directions, i.e., determining if the known antibody blocks the test antibody and vice versa. The competition experiments may be performed, e.g., using a Biacore® T200 instrument.

Antigen-binding portions of the anti-hIgG antibodies disclosed herein may be used in lieu of full antibodies. The term “antigen-binding portion” refers to one or more portions or fragments of an antibody that retain the ability to specifically bind to an antigen (e.g., human IgG, or a fragment thereof). Examples of antigen-binding portions are, without limitation, (i) a Fab fragment: a monovalent fragment consisting of the VL, VH, CL and CH1 domains; (ii) a F(ab′)₂ fragment: a bivalent fragment comprising two Fab fragments linked by a disulfide bridge at the hinge region; (iii) an Fd fragment consisting of the VH and CH1 domains; (iv) an Fv fragment consisting of the VL and VH domains of a single arm of an antibody; (v) a dAb fragment, which consists of a VH domain; and (vi) an isolated complementarity determining region (CDR) capable of specifically binding to an antigen. Furthermore, although the two domains of the Fv fragment, VL and VH, are encoded by separate genes, they can be joined, using recombinant methods, by a synthetic linker that enables them to be made as a single protein chain in which the VL and VH domains pair to form monovalent molecules (known as single chain Fv (scFv)). Other forms of single chain antibodies, such as diabodies, are also encompassed. Diabodies are bivalent, bi-specific antibodies in which VH and VL domains are expressed on a single polypeptide chain, but using a linker that is too short to allow for pairing between the two domains on the same chain, thereby forcing the domains to pair with complementary domains of another chain and creating two antigen-binding sites. Antibody portions, such as Fab and F(ab′)₂ fragments, can be prepared from whole antibodies using conventional techniques, such as papain or pepsin digestion of whole antibodies, or recombinant DNA techniques. The present antibodies bind to one, more, or all of human IgG subtypes hIgG1, hIgG2, hIgG3, and hIgG4. In certain embodiments, it binds all of said subtypes. It is understood that the antibodies described herein may also bind to humanized and/or chimeric antibodies comprising sequences from human IgG.

In some embodiments, the present disclosure provides an anti-hIgG monoclonal antibody or an antigen-binding portion thereof whose heavy chain CDR1-3 and light chain CDR1-3 comprise SEQ ID NOs: 27-32, 33-38, 39-44, 45-50, 51-56, or 57-62, respectively. The framework of this antibody may be derived from an antibody from a rabbit or another species (e.g., mouse, human, or rat).

In some embodiments, the present disclosure provides an anti-hIgG monoclonal antibody or an antigen-binding portion thereof whose heavy chain variable domain (VH) and light chain variable domain (VL) comprise SEQ ID NOs: 13 and 19, 14 and 20, 15 and 21, 16 and 22, 17 and 23, or 18 and 24, respectively. The constant region of this antibody may be derived from an antibody from rabbit or another species (e.g., mouse, human, or rat).

In some embodiments, the present disclosure provides an anti-hIgG monoclonal antibody comprising:
a) a heavy chain (HC) comprising the amino acid sequences of SEQ ID NOs: 13 and 25 and a light chain (LC) comprising the amino acid sequences of SEQ ID NOs: 19 and 26;
b) an HC comprising the amino acid sequences of SEQ ID NOs: 14 and 25 and an LC comprising the amino acid sequences of SEQ ID NOs: 20 and 26;
c) an HC comprising the amino acid sequences of SEQ ID NOs: 15 and 25 and an LC comprising the amino acid sequences of SEQ ID NOs: 21 and 26;
d) an HC comprising the amino acid sequences of SEQ ID NOs: 16 and 25 and an LC comprising the amino acid sequences of SEQ ID NOs: 22 and 26;
e) an HC comprising the amino acid sequences of SEQ ID NOs: 17 and 25 and an LC comprising the amino acid sequences of SEQ ID NOs: 23 and 26; or
f) an HC comprising the amino acid sequences of SEQ ID NOs: 18 and 25 and an LC comprising the amino acid sequences of SEQ ID NOs: 24 and 26.

In some embodiments, the anti-hIgG antibody or antigen-binding portion has a VH amino acid sequence that is at least 80%, 85%, 90%, 91%, 92%, 93%, 94%, 95%, 96%, 97%, 98%, or 99% identical to the amino acid sequence of SEQ ID NO: 13, 14, 15, 16, 17, or 18.

In some embodiments, the anti-hIgG antibody or antigen-binding portion has a VL amino acid sequence that is at least 80%, 85%, 90%, 91%, 92%, 93%, 94%, 95%, 96%, 97%, 98%, or 99% identical to the amino acid sequence of SEQ ID NO: 19, 20, 21, 22, 23, or 24.

In some embodiments, the anti-hIgG antibody or antigen-binding portion has VH and VL amino acid sequences that are at least 80%, 85%, 90%, 91%, 92%, 93%, 94%, 95%, 96%, 97%, 98%, or 99% identical, to SEQ ID NOs: 13 and 19, 14 and 20, 15 and 21, 16 and 22, 17 and 23, or 18 and 24, respectively.

In some embodiments, the anti-hIgG antibody has a HC and a LC comprising SEQ ID NOs: 63 and 69, 64 and 70, 65 and 71, 66 and 72, 67 and 73, or 68 and 74, respectively, with or without the leader sequence.

In some embodiments, the anti-hIgG antibody or antigen-binding portion of the present disclosure comprises the HCDR1-3 and LCDR1-3, VH and VL, or HC and LC amino acid sequences of antibody 2C5, 9E6, 11F9, 11G5, 16F5, or 19B1.

The assignment of amino acid numbers and of CDRs may be in accordance with the definitions of Kabat, Sequences of Proteins of Immunological Interest (National Institutes of Health, Bethesda, MD (1987 and 1991)). See also Zhang, supra.

An anti-hIgG antibody or antigen-binding portion of the present disclosure can be derivatized or linked to another molecule (e.g., another peptide or protein). In general, the antibodies or portions thereof are derivatized such that IgG binding is not affected adversely by the derivatization or labeling. For example, an antibody or antibody portion of the present disclosure can be functionally linked (by chemical coupling, genetic fusion, noncovalent association or otherwise) to one or more other molecular entities, such as another antibody or a detectable label or tag. Examples include, but are not limited to, radioisotopes or radionuclides (e.g., ³H, ¹⁴C, ¹⁵N, ³⁵S, ⁹⁰Y, ⁹⁹Tc, ¹¹¹In, ¹²⁵I, ¹³¹I), fluorescent labels (e.g., FITC, rhodamine, lanthanide phosphors, phycoerythrin, or the Alexa Fluor® dyes), enzymatic labels (e.g., horseradish peroxidase, β-galactosidase, luciferase, alkaline phosphatase), chemiluminescent markers, biotinyl groups, predetermined polypeptide epitopes recognized by a secondary reporter (e.g., leucine zipper pair sequences, binding sites for secondary antibodies, metal binding domains, epitope tags), and magnetic agents such as gadolinium chelates. In some embodiments, labels are attached by spacer arms of various lengths to reduce potential steric hindrance.

The present anti-hIgG antibodies and antigen-binding portions of the present disclosure are useful for detecting and/or measuring the level of human IgG or Fab in a sample from an animal (e.g., a non-human primate such as a cynomolgus or rhesus monkey). In some embodiments, the antibodies and antigen-binding portions can be used to detect and/or measure the level of human IgG or Fab in a sample from a human. Suitable detection and measurement methods include immunological methods such as enzyme-linked immunosorbent assays (ELISA), radioimmuno assays, and immunohistology. In some embodiments, the antibodies and antigen-binding portions can be used to detect and/or measure the level of human IgG or Fab in a sample from a human for preclinical or clinical immunohistochemistry (IHC) studies.

Because the rabbit antibodies described herein can bind to distinct epitopes, they can be used alone or used in pairs for the detection of human IgG, in any host animal, to meet the need for therapeutic monoclonal antibody development in preclinical studies. For example, antibodies 16F5, 11F9, and 11G5/19B1/2C5/9E6 bind to three distinct epitopes that are different also from the epitope of MCA5748G. Thus, a pair selected from them that bind to two distinct epitopes may be used together to, e.g., increase assay sensitivity and specificity. For example, 16F5 may be used together with 11F9; 16F5 or 11F9 may be used together with 11G5, 19B1, 2C5, or 9E6; and MCA5748G can used with any one of 16F5, 11F9, 11G5, 19B1, 2C5, and 9E6. The antibodies in the pair may be labeled differently.

### Production of the Anti-hIgG Antibodies

The present anti-hIgG antibodies may be produced by well-known hybridoma technology, in which rabbit B cells producing the antibody of interest are fused with an immortalized cell to form hybridoma cell lines that produce the antibody.

Alternatively, the present hIgG antibodies or antigen-binding portions thereof are produced by recombinant technology using host cells containing nucleotide sequences encoding the heavy and light chains of the antibodies or portions. Accordingly, the present disclosure also provides nucleic acid molecules and sequences encoding anti-IgG antibodies or antigen-binding portions thereof described herein. The nucleotide sequences encoding the heavy chain and light chain amino acid sequences may be introduced to the host cells in two different vectors or on the same vector. They may be expressed under the transcriptional control of a single promoter or two separate promoters.

In some embodiments, the present nucleic acid molecules comprise nucleotide sequences that are at least 70%, 75%, 80%, 85%, 90%, 95%, 97%, 98% or 99% identical to (i) SEQ ID NO: 1, 2, 3, 4, 5, 6, 7, 8, 9, 10, 11, or 12; or (ii) a nucleotide sequence encoding SEQ ID NO: 13, 14, 15, 16, 17, 18, 19, 20, 21, 22, 23, 24, 25, or 26.

The term “percent sequence identity” in the context of amino acid and nucleic acid sequences refers to the residues in two sequences that are the same when aligned for maximum correspondence. For example, the length of sequence identity comparison may be over a stretch of at least about nine nucleotides, usually at least about 18 nucleotides, more usually at least about 24 nucleotides, typically at least about 28 nucleotides, more typically at least about 32 nucleotides, and preferably at least about 36, 48 or more nucleotides. There are a number of different algorithms known in the art which can be used to measure amino acid and nucleotide sequence identity. For instance, polynucleotide sequences can be compared using FASTA, Gap or Bestfit, which are programs in Wisconsin Package Version 10.0, Genetics Computer Group (GCG), Madison, Wisconsin. FASTA, which includes, e.g., the programs FASTA2 and FASTA3, provides alignments and percent sequence identity of the regions of the best overlap between the query and search sequences (see, e.g., Pearson, Methods Enzymol. (1990) 183:63-98; Pearson, Methods Mol. Biol. (2000) 132:185-219; Pearson, Methods Enzymol. (1996) 266:227-58; and Pearson, J. Mol. Biol. (1998) 276:71-84; incorporated herein by reference). Unless otherwise specified, default parameters for a particular program or algorithm are used. For instance, percent sequence identity between nucleic acid sequences can be determined using FASTA with its default parameters (a word size of 6 and the NOPAM factor for the scoring matrix) or using Gap with its default parameters as provided in GCG Version 6.1, incorporated herein by reference.

In particular embodiments, the present disclosure provides a nucleic acid molecule comprising a nucleotide sequence selected from the group consisting of SEQ ID NOs: 1, 2, 3, 4, 5, 6, 7, 8, 9, 10, 11, and 12. In certain embodiments, the nucleic acid molecule comprises the nucleotide sequences of SEQ ID NOs: 1 and 7, 2 and 8, 3 and 9, 4 and 10, 5 and 11, or 6 and 12.

In any of the above embodiments, the nucleic acid molecules may be isolated. Nucleic acid molecules referred to herein as “isolated” or “purified” are nucleic acids which (1) have been separated away from the nucleic acids of the genomic DNA or cellular RNA of their source of origin; and/or (2) do not occur in nature.

In a further aspect, the present disclosure provides a vector suitable for expressing one or both of the chains of an antibody or antigen-binding portion thereof as described herein. The term “vector,” as used herein, means a nucleic acid molecule capable of transporting another nucleic acid to which it has been linked. In some embodiments, the vector is a plasmid, i.e., a circular double stranded piece of DNA into which additional DNA segments may be ligated. Moreover, certain vectors are capable of directing the expression of genes to which they are operatively linked. Such vectors are referred to herein as “recombinant expression vectors” (or simply, “expression vectors”).

The present disclosure provides vectors comprising nucleic acid molecules that encode the heavy chain, the light chain, or both the heavy and light chains of an anti-hIgG antibody as described herein or an antigen-binding portion thereof. The vector may further comprise an expression control sequence.

The term “expression control sequence” as used herein means polynucleotide sequences that are necessary to affect the expression and processing of coding sequences to which they are ligated. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include, at a minimum, all components whose presence is essential for expression and processing, and can also include additional components whose presence is advantageous, for example, leader sequences and fusion partner sequences.

In some embodiments, a nucleic acid molecule as described herein comprises a nucleotide sequence encoding a VH domain from an anti-IgG antibody or antigen-binding portion as described herein joined in-frame to a nucleotide sequence encoding a heavy chain constant region from any source. Similarly, a nucleic acid molecule as described herein can comprise a nucleotide sequence encoding a VL domain from an anti-IgG antibody or antigen-binding portion as described herein joined in-frame to a nucleotide sequence encoding a light chain constant region from any source.

In a further aspect of the present disclosure, nucleic acid molecules encoding the VH and/or VL may be “converted” to full-length antibody genes. In some embodiments, nucleic acid molecules encoding the VH or VL domains are converted to full-length antibody genes by insertion into an expression vector already encoding heavy chain constant (CH) or light chain constant (CL) regions, respectively, such that the VH segment is operatively linked to the CH segment(s) within the vector, and/or the VL segment is operatively linked to the CL segment within the vector. In another aspect, nucleic acid molecules encoding the VH and/or VL domains are converted into full-length antibody genes by linking, e.g., ligating, a nucleic acid molecule encoding a VH and/or VL domain to a nucleic acid molecule encoding a CH and/or CL region using standard molecular biological techniques. Nucleic acid molecules encoding the full-length heavy and/or light chains may then be expressed from a cell into which they have been introduced and the anti-IgG antibody isolated.

In some embodiments, the framework region(s) are mutated so that the resulting framework region(s) have the amino acid sequence of the corresponding germline gene. A mutation may be made in a framework region or constant region, e.g., to increase the half-life of the anti-IgG antibody. See, e.g., PCT Publication WO 00/09560. A mutation in a framework region or constant region also can be made to alter the immunogenicity of the antibody, and/or to provide a site for covalent or non-covalent binding to another molecule. According to the present disclosure, an antibody may have mutations in any one or more of the CDRs or framework regions of the variable domain or in the constant region.

The present disclosure also provides methods for producing the antibody compositions and antibodies and antigen-binding portions thereof described herein. In some embodiments, the present disclosure relates to a method for producing an anti-IgG antibody or antigen-binding portion as described herein, comprising providing a recombinant host cell comprising a nucleotide sequence that encodes the heavy chain or an antigen-binding portion thereof, and a nucleotide sequence that encodes the light chain or an antigen-binding portion thereof, of an anti-IgG antibody or antigen-binding portion described herein; cultivating said host cell under conditions suitable for expression of the antibody or antigen-binding portion; and isolating the resulting antibody or antigen-binding portion. Antibodies or antigen-binding portions produced by such expression in such recombinant host cells are referred to herein as “recombinant” antibodies or antigen-binding portions. The present disclosure also provides progeny cells of such host cells, and antibodies or antigen-binding portions produced by same.

The term “recombinant host cell” (or simply “host cell”), as used herein, means a cell into which a recombinant expression vector has been introduced. By definition, a recombinant host cell does not occur in nature. The present disclosure provides host cells that may comprise, e.g., a vector as described herein. The present disclosure also provides host cells that comprise, e.g., a nucleotide sequence encoding the heavy chain or an antigen-binding portion thereof, a nucleotide sequence encoding the light chain or an antigen-binding portion thereof, or both, of an anti-IgG antibody or antigen-binding portion thereof described herein. It should be understood that “recombinant host cell” and “host cell” mean not only the particular subject cell but also the progeny of such a cell. Because certain modifications may occur in succeeding generations due to either mutation or environmental influences, such progeny may not, in fact, be identical to the parent cell, but are still included within the scope of the term “host cell” as used herein.

Nucleic acid molecules encoding anti-IgG antibodies and antigen-binding portions thereof and vectors comprising these nucleic acid molecules can be used for transfection of a suitable mammalian, plant, bacterial or yeast host cell. Transformation can be by any known method for introducing polynucleotides into a host cell. Methods for introduction of heterologous polynucleotides into mammalian cells are well known in the art and include dextran-mediated transfection, calcium phosphate precipitation, polybrene-mediated transfection, protoplast fusion, electroporation, encapsulation of the polynucleotide(s) in liposomes, and direct microinjection of the DNA into nuclei. In addition, nucleic acid molecules may be introduced into mammalian cells by viral vectors.

It is likely that antibodies expressed by different cell lines or in transgenic animals will have different glycosylation patterns from each other. However, all antibodies encoded by the nucleic acid molecules provided herein, or comprising the amino acid sequences provided herein are part of the present disclosure, regardless of the glycosylation state of the antibodies, and more generally, regardless of the presence or absence of post-translational modification(s).

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Generally, nomenclature used in connection with, and techniques of, cell and tissue culture, molecular biology, immunology, microbiology, genetics, analytical chemistry, synthetic organic chemistry, medicinal and pharmaceutical chemistry, and protein and nucleic acid chemistry and hybridization described herein are those well-known and commonly used in the art. Enzymatic reactions and purification techniques are performed according to manufacturer's specifications, as commonly accomplished in the art or as described herein. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety. Although a number of documents are cited herein, this citation does not constitute an admission that any of these documents form part of the common general knowledge in the art.

In order that the present disclosure may be better understood, the following examples are set forth. These examples are for purposes of illustration only and are not to be construed as limiting the scope of the present disclosure in any manner.

### EXAMPLES

The following examples describe experiments in which a human IgG Fab was used to immunize rabbits and the splenocytes were isolated and used to sort human IgG-specific B-cells. Using cynomolgus monkey IgG as counter screening agent, we obtained six human IgG and Fab specific mAb clones. These clones demonstrated better binding affinity and targeted different epitopes from the only commercially available mouse-origin anti-hIgG mAb clone MCA5748G. These rabbit anti-hIgG mAb clones were evaluated by Gyrolab™ assays and found to be suitable for use as capture reagent in generic pharmacokinetic assays in the presence of cynomolgus monkey serum. The materials and methods for the experiments described herein are as follows.

### Chemicals and Reagents

The therapeutic antibodies used in these experiments were a humanized therapeutic monoclonal antibody IgG1 (hIgG1), a humanized development candidate monoclonal antibody IgG4 (hIgG4) and an internal research reagent human Fab (hFab). Cynomolgus monkey IgG (cynoIgG) was purified from cynomolgus monkey serum, purchased from Innovative Research (Novi, MI 48377), by protein A affinity purification. The mouse anti-human IgG monoclonal antibody MCA5748G (Stubenrauch et al., J Pharm Biomed Anal. (2009) 49:1003-8) was purchased from BioRad Laboratories (Hercules, CA). Cell culture medium and phosphate-buffered saline (PBS) were purchased from ThermoFisher Scientific (Waltham, MA). All other chemicals were analytical grade.

### ELISA for Determination of Binding Specificity

Enzyme-linked immunosorbent assay (ELISA) was used to evaluate the specificity of the antibody clones for binding to human immunoglobulin (IgG) using cynoIgG as control. The ELISA was performed at room temperature on microtiter plates from ThermoFisher Scientific (Waltham, MA), which were first coated with hIgG1, hIgG4 or cynoIgG in PBS for 1 hr. After washing three times with phosphate-buffered saline-polysorbate 20 (Tween 20), the plate was blocked with PBS/3% bovine serum albumin for 1 h. The plate was then washed again and incubated with anti-human IgG antibody clones for 1 h. After another washing step, the bound antibodies were detected by horseradish peroxidase (HRP)-conjugated anti-rabbit IgG antibody from Southern Biotech (Birmingham, AL), according to manufacturer's instructions.

### Biacore Assay for Determination of Binding Specificity and Kinetics

The specificity of the rabbit anti-human IgG monoclonal antibody was evaluated in a second assay system as described elsewhere (Chu et al., Sci Rep. (2015) 4:7360). These experiments were performed with the Biacore® T200 instrument (Biacore, Uppsala, Sweden) using Streptavidin or CM5 sensor chips from Biacore. Coating of an antibody to streptavidin chip was achieved by injecting biotinylated target antibody, which was amine coupled using the EZ-Link™ Amine-PEG11-Biotin reagent from ThermoFisher Scientific (Waltham, MA) following manufacture's manual. For the CM5 chip, the target antigen or antibody was coupled to the chip surface, via standard amine coupling, using the amine coupling kit from Biacore. Unless otherwise stated, all binding and kinetics assays were performed in HBS-EP+ buffer (0.01 M HEPES pH 7.4, 0.15 M NaCl, 3 mM EDTA, 0.005% v/v Surfactant P20) at 25° C. The dissociation constant values (KD) were calculated with a 1:1 Langmuir fitting model using BIAevaluation software V4.1 from Biacore.

### Rabbit Immunization and B-Cell Cloning

The hIgG Fab, as described by Wu et al., Nat Cancer (2020) 1:86-98, was used to immunize two rabbits with the total of five antigen injections. The primary injection used complete Freund's adjuvant (CFA) and the four boosts used incomplete Freund's adjuvant (IFA). The CFA and IFA were both from ThermoFisher Scientific (Waltham, MA). Serum titers were monitored by ELISA using the antigen protein. The rabbit with higher ELISA titer was chosen for splenectomy.

For B-cell isolation, fresh splenocytes were isolated from the spleen. Approximately 1.2×10⁸ splenocytes were cultured overnight in a special B cell medium customized by Yurogen (Worcester, MA) before sorting. Splenocytes were processed using SMab™ platform at Yurogen to enrich antigen-recognizing B cells. The antigen-sorted B cells were seeded and cultured in a 96-well plate, with 1 cell/well, for 10-14 days.

Antigen-recognizing B-cell clones were identified and confirmed using hIgG4 coated direct ELISA, purified cynoIgG was used in ELISA for counter screening. Antigen-specific B-cell clones were ranked and selected according to the positive/negative ELISA signal ratio and their heavy and light chains of IgG coding sequences were amplified by RT-PCR. The heavy and light chain PCR products were combined and used to transfect HEK293F cells directly. Transiently expressed recombinant rabbit IgG clones were then further confirmed for specific binding to hIgG1, hIgG4 and hFab by ELISA and Biacore binding assays. Upon confirming specific binding to hIgG1, hIgG4 and hFab, the PCR products from the selected positive B-cells were cloned into mammalian expression vector for scale up antibody production in HEK293F cells. Recombinant rabbit mAb clones produced by HEK293F transfection were purified using protein A chromatography for further evaluation.

### Gyrolab™ Assay

Gyrolab™ xPlore, Bioaffy 1000 nL CDs, Rexxip A and Rexxip F Buffer from Gyros Protein Technologies (Uppsala Sweden) were used for all experiments (Fraley et al., Bioanalysis (2013) 5:1765-74). Biotinylated capture antibodies were diluted to 0.1-0.2 μg/μL in Rexxip A Buffer and flowed over the streptavidin bead column within the microstructure of the Bioaffy CDs. The standard curve and quality control (QC) samples were prepared by spiking the hIgG4 or hFab at the range as indicated in Rexxip A Buffer containing various amount of cynomolgus monkey serum. The standard curve samples, QC samples, mock samples and assay reagents were added to PCR plates and loaded onto the Gyrolab™ instrument. A single replicate of the standard curve, QC samples or mock samples were added into two CD microstructures by the Gyros instrument and then flowed over the bead column. Alexa fluor 647-labeled goat anti-human IgG (Fc) antibody purchased from Southern Biotech (Birmingham, AL) was used as the detection reagent at 2 μg/ml in Rexxip F buffer. A wash solution of PBS with 0.01% v/v Tween-20 was flowed over the columns prior to each run in order to pre-wet the streptavidin beads, and after each step in the assay, to rinse away any unbound reagents. Sample concentrations were determined with the data acquisition at a 1% level of photomultiplication. The Gyrolab™ Evaluator Program was used to analyze results with a 5-parameter fit and 1/Y2 weighting as directed by the manufacture.

### Example 1: Isolation of Human IgG-Specific Rabbit Antibody Clones

This example describes experiments in which six rabbit antibody clones recognizing both human IgG and Fab but not cynomolgus monkey IgG were developed using rabbit B-cell cloning technology.

Although hybridoma screening and display methodologies have been used in rabbit monoclonal antibody development, they both have some drawbacks: the hybridoma technology has a low efficiency of cell fusion, while display method results in the loss of natural cognate pairing of heavy and light chain (Zhang et al., Front Immunol. (2017) 8:494). To overcome these issues, a single B cell-based antibody gene cloning technology (or single B cell cloning) has been developed recently (Seeber et al., PLOS ONE (2014) 9:e86184; Rashidian et al., “Single B Cell Cloning and Production of Rabbit Monoclonal Antibodies” in: Zielonka and Krah (eds) Genotype Phenotype Coupling. Methods in Molecular Biology, vol 2070, Humana, New York, NY, 2020).

Briefly, the single B cell cloning consists of the following steps: (i) isolating specific single B cells from peripheral blood or from lymphoid tissues by antigen-based FACS sorting, (ii) growing and expanding the single B cell for two weeks, (iii) performing RT-PCR with antibody-specific primers to amplify antibody genes and sequencing, (iv) cloning the antibody genes into an expression vector and produce recombinant monoclonal antibody in mammalian cell systems (e.g., HEK 293, CHO cells), and (v) purifying and evaluating recombinant monoclonal antibody by ELISA and other in vitro assays.

We used the human IgG1 Fab to immunize rabbits and the resultant splenocytes were sorted by biotinylated hIgG1, which was a humanized whole IgG1 molecule. A total of 530 primary B cells were single-cell seeded into 96-well plates and grown for two weeks. The B-cell culture medium, which contained monoclonal rabbit IgG antibody, was screened by direct ELISA using hIgG4, hIgG1 and cynoIgG. Based on the ELISA hIgG1/cynoIgG and hIgG4/cynoIgG signal values, we picked 17 clones that had both hIgG1 and hIgG4 OD450>0.9 and CynoIgG OD450<0.2 (Table 1), and their antibody coding sequences were amplified by PCR.

TABLE 1
ELISA screen of PCR product-transfected cell culture medium

| | hIgG4/CynoIgG ELISA | | hIgG1/CynoIgG ELISA | |
|---|---|---|---|---|
| | hIgG4 | CynoIgG | hIgG1 | CynoIgG |
| Antigen | (OD450 > 0.9) | (OD450 < 0.2) | (OD450 > 0.9) | (OD450 < 0.2) |
| 16F5 | 1.374 | 0.154 | 1.394 | 0.184 |
| 11G5 | 1.317 | 0.159 | 1.254 | 0.161 |
| 14G6 | 1.311 | 0.169 | 1.583 | 0.141 |
| 2C5 | 1.29 | 0.124 | 1.243 | 0.107 |
| 9E6 | 1.282 | 0.146 | 1.281 | 0.14 |
| 2G11 | 1.203 | 0.083 | 1.375 | 0.073 |
| 12C10 | 1.093 | 0.071 | 1.13 | 0.097 |
| 18C1 | 0.984 | 0.094 | 0.929 | 0.093 |
| 4E5 | 0.961 | 0.093 | 1.201 | 0.08 |
| 2D2 | 0.959 | 0.095 | 0.963 | 0.066 |
| 2G3 | 0.957 | 0.082 | 1.27 | 0.087 |
| 9H11 | 0.957 | 0.074 | 1.029 | 0.068 |
| 19B1 | 0.954 | 0.102 | 1.149 | 0.055 |
| 17E8 | 0.946 | 0.086 | 1.251 | 0.064 |
| 2A7 | 0.945 | 0.101 | 1.036 | 0.046 |
| 20D5 | 0.928 | 0.107 | 1.072 | 0.074 |
| 11F9 | 0.91 | 0.092 | 1.109 | 0.089 |

In eleven of these 17 clones, we were able to obtain PCR products for both heavy and light chains. The heavy and light chain PCR products from each clone were combined at a 1:1 ratio and used to transfect HEK293F cells directly. Cell culture medium from the transfected HEK293F cells was further confirmed by the same ELISA screen assay. Table 2 shows the binding of six of the rabbit recombinant antibody clones with specific binding to whole human IgG (hIgG) and Fab, but not to cynomolgus monkey IgG (cynoIgG), as indicated by the signal ratio of their binding to hIgG1 or hIgG4 versus cynoIgG determined by ELISA assay. These six clones exhibited good hIgG4/cynoIgG and hIgG1/cynoIgG ELISA signal ratio ranging from 4.94 to 14.99.

TABLE 2
Exemplary Rabbit mAbs

| | Selected Clones | | | | | |
|---|---|---|---|---|---|---|
| ELISA Signal Ratio | 2C5 | 9E6 | 11F9 | 11G5 | 16F5 | 19B1 |
| hIgG4/CynoIgG | 7.91 | 9.53 | 5.92 | 5.19 | 6.33 | 12.01 |
| hIgG1/CynoIgG | 7.72 | 10.17 | 6.20 | 4.94 | 5.90 | 14.99 |

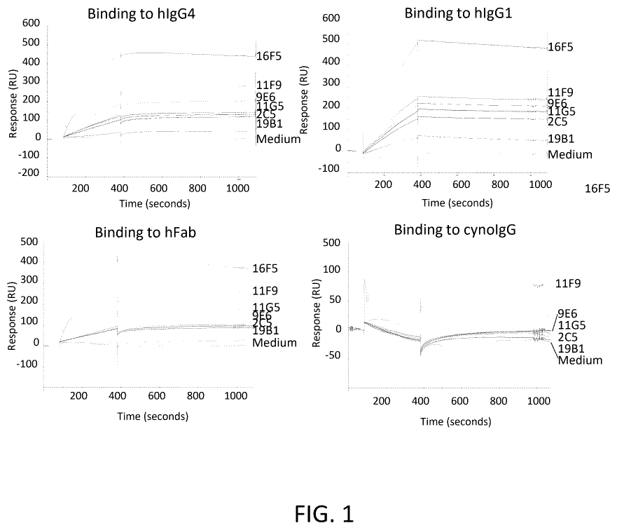

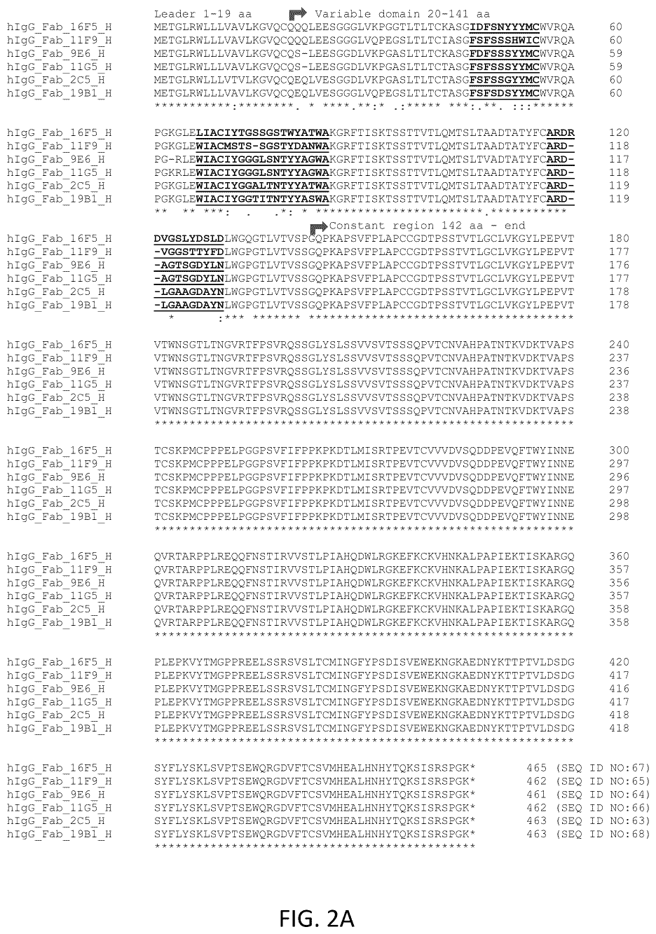

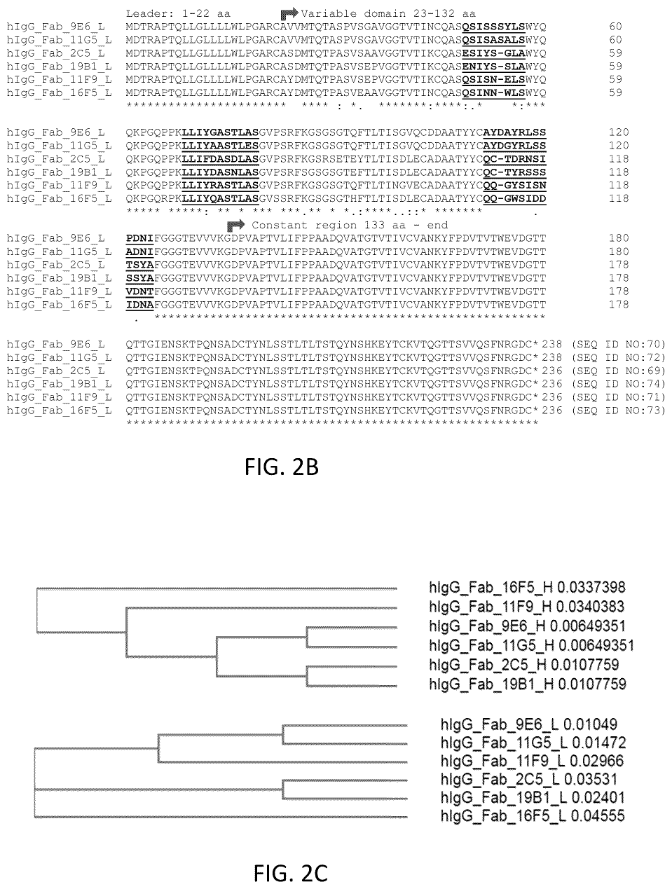

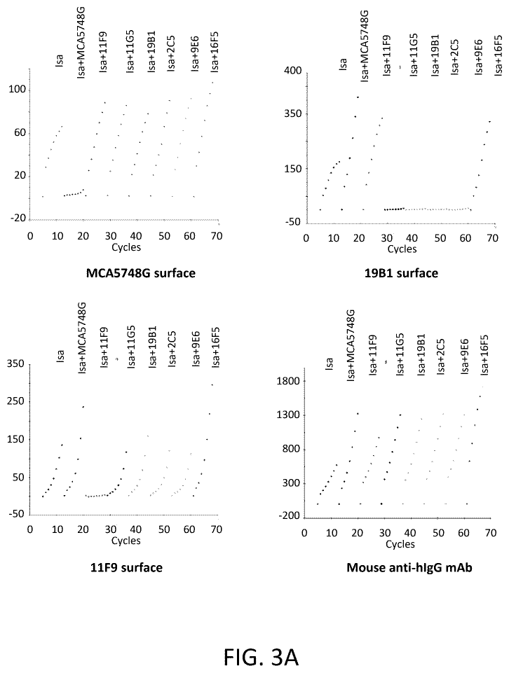

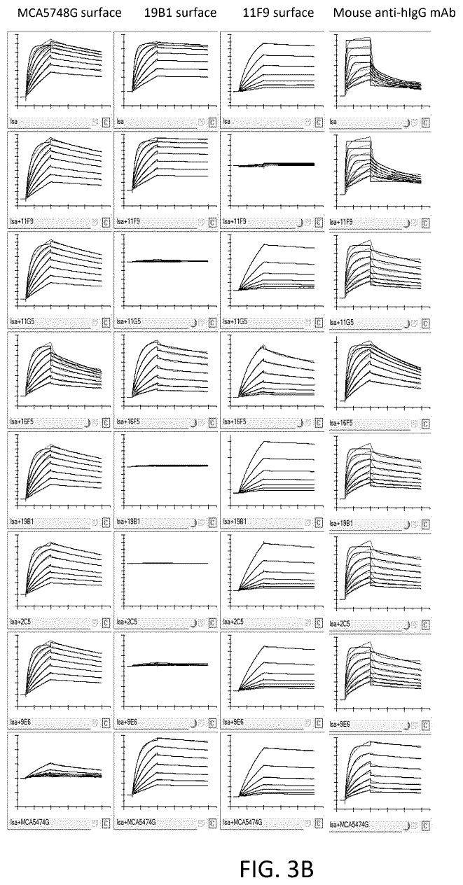

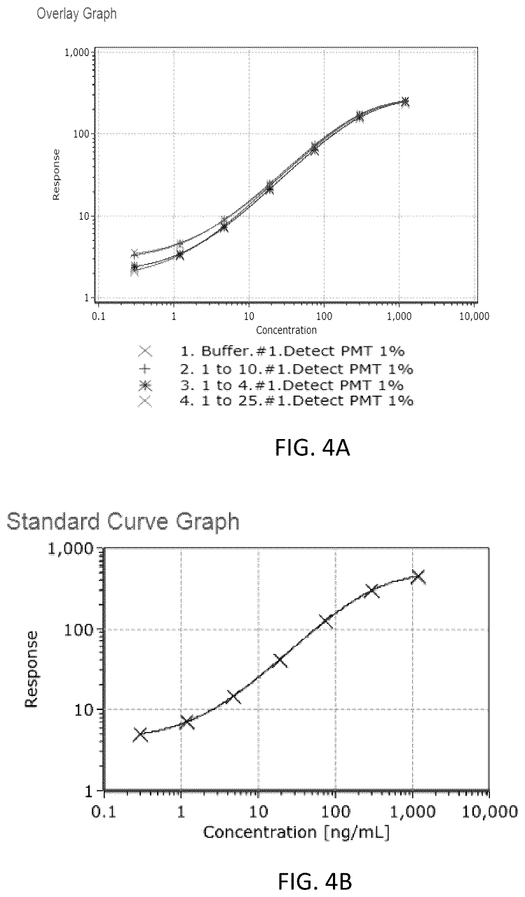

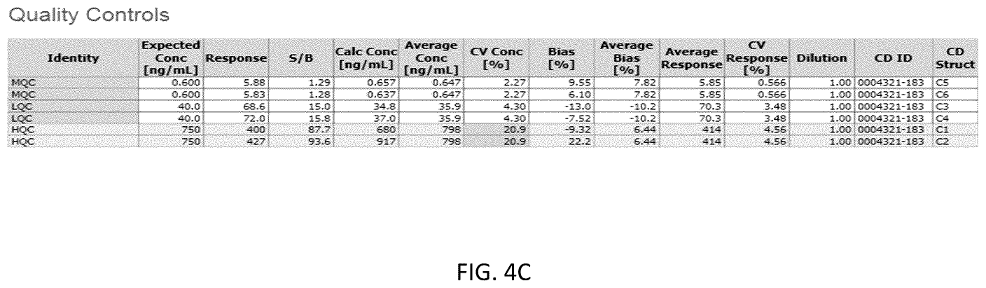

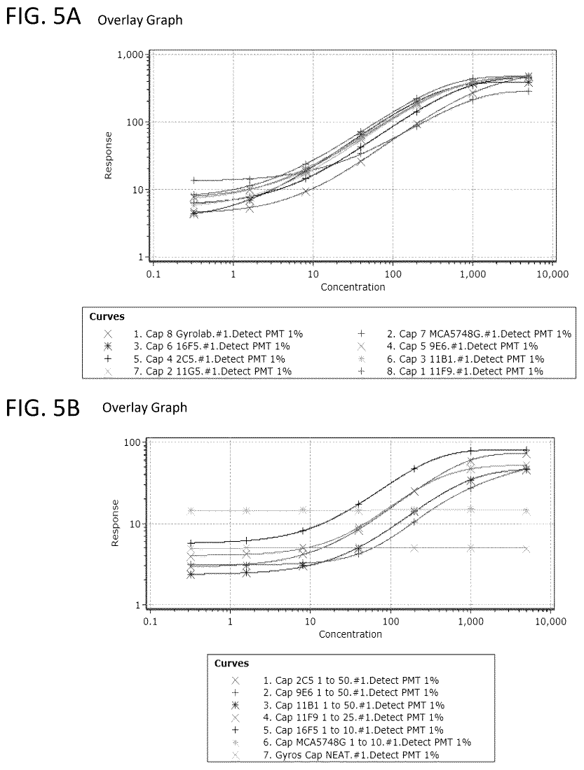

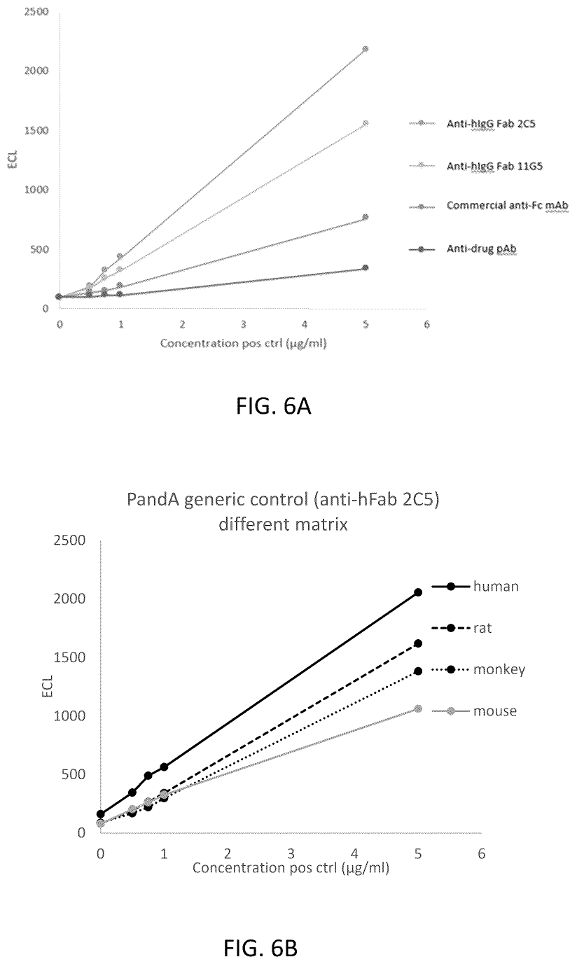

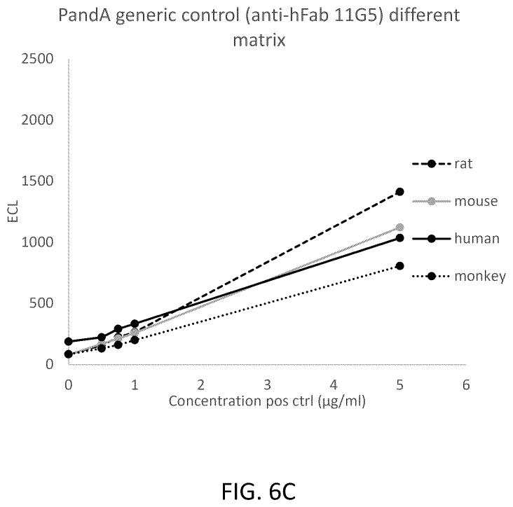

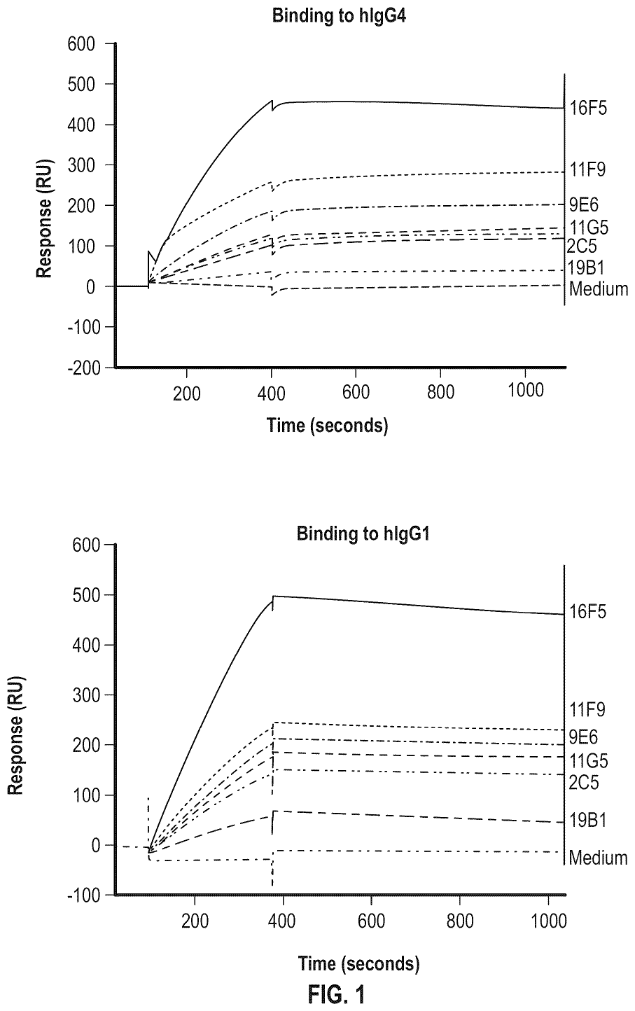

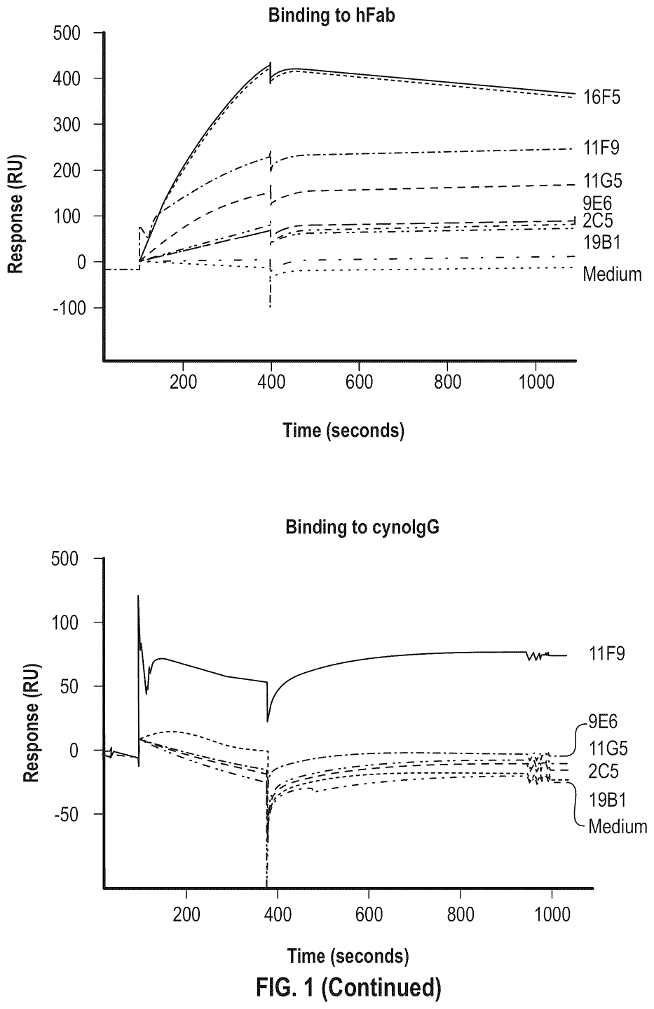

These six clones were further analyzed for direct binding to hIgG1, hIgG4, hFab and cynoIgG by Biacore as shown in FIG. 1. All six clones showed various levels of binding to hIgG1, hIgG4 and hFab, but not to cynoIgG. These data indicate that these clones are specific to human IgGs and Fab, but not to cynoIgG.

### Example 2: Sequences of Rabbit Anti-hIgG Antibodies

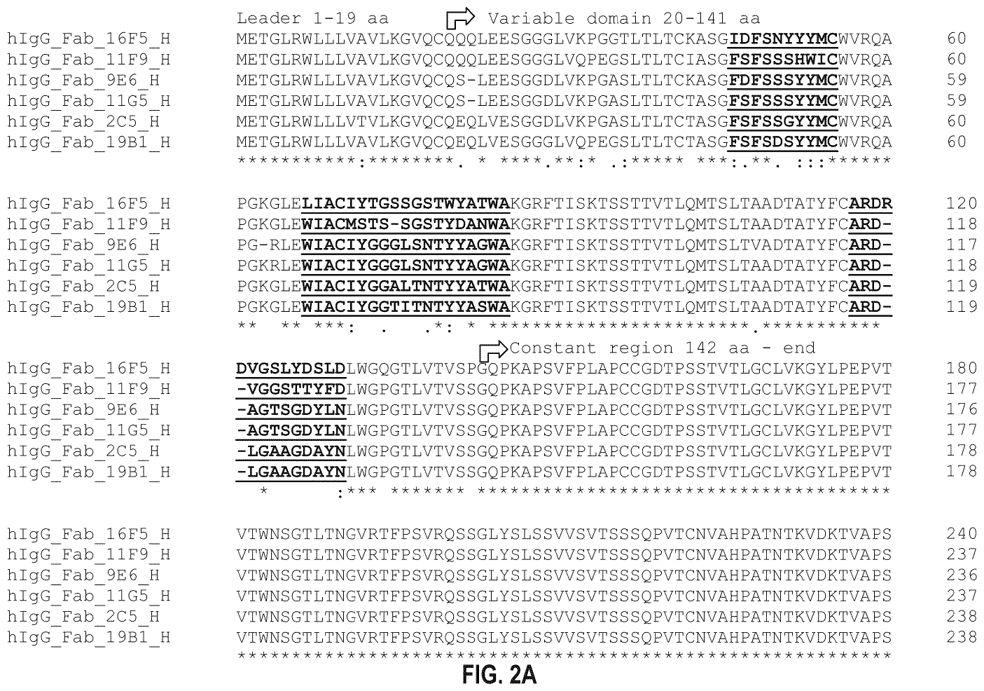

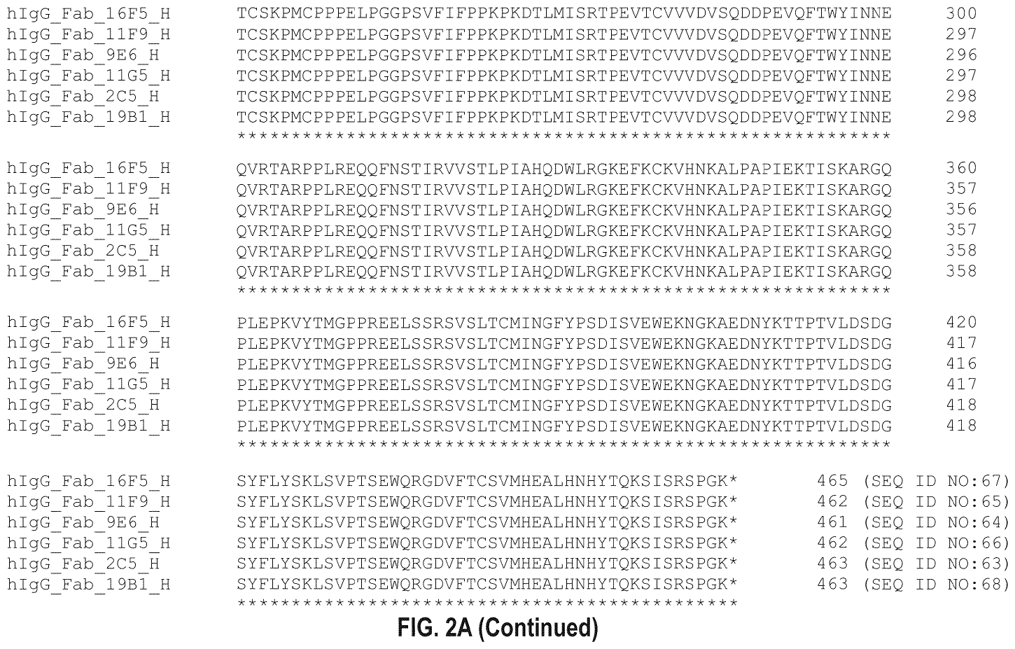

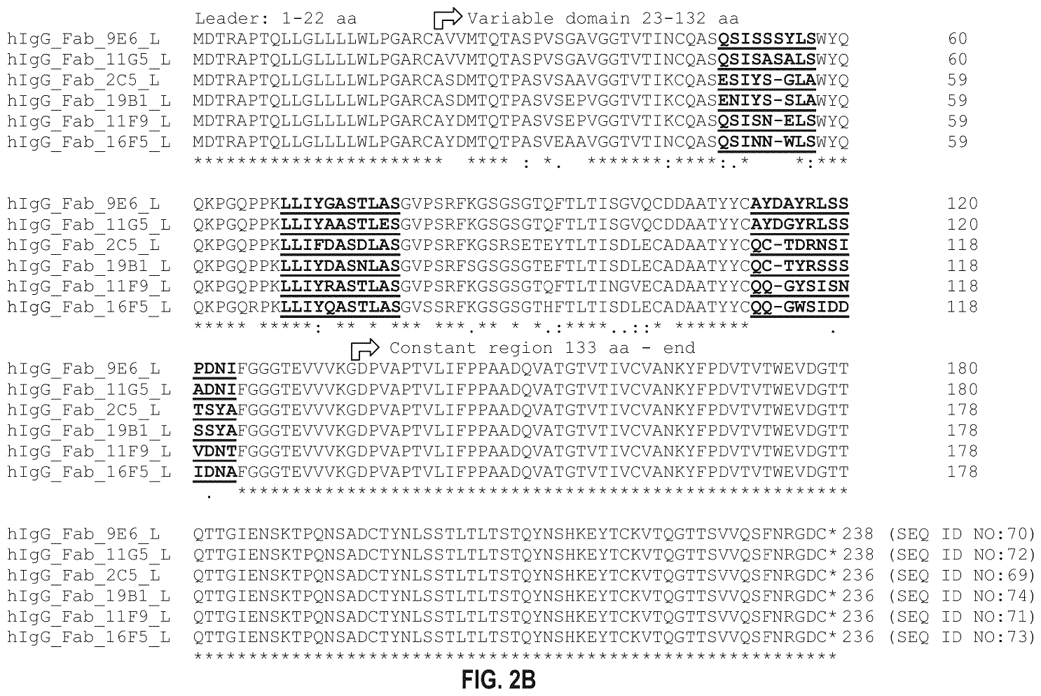

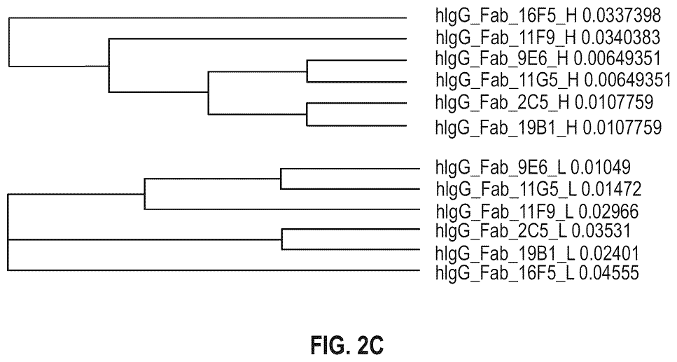

In order to determine the uniqueness of the six clones, heavy and light chain PCR products were subjected to DNA sequencing. The deduced amino acid sequences of the variable regions for the heavy and light chains, respectively, were aligned using the EMBL-EBI web based Clustal Omega (Sievers et al., Mol Syst Biol. (2011) 7:539). As shown in FIGS. 2A and 2B, all six clones possess unique amino acid sequences for both heavy and light chains. The phylogenetic trees, based on Clustal Omega, were constructed to visualize relative distances (FIG. 2C). Notably, clone 16F5 is highly divergent from other five clones in both heavy and light chains, whereas clone 11F9 is the next divergent clone in the heavy chain but not in the light chain.

Table 3 shows the nucleotide sequences encoding antibodies 205, 9E6, 11F9, 11G5, 16F5, and 19B1 (SEQ: SEQ ID NO).

TABLE 3
Nucleotide sequences of exemplary mAbs

| Ab Chain | Nucleotide Sequence (5′ to 3′) | SEQ |
|---|---|---|
| 2C5 H | ATGGAGACTGGGCTGCGCTGGCTTCTCCTGGTCACTGTGCTCAAAGGTGTCCA | 1 |
| | GTGTCAGGAGCAGCTGGTGGAGTCCGGGGGAGACCTGGTCAAGCCTGGGGCAT | |
| | CCCTGACACTCACCTGCACAGCCTCTGGATTCTCCTTCAGTAGCGGCTACTAC | |
| | ATGTGCTGGGTCCGCCAGGCTCCAGGGAAGGGGCTGGAGTGGATCGCATGCAT | |
| | TTATGGTGGTGCGCTTACTAATACTTACTACGCGACCTGGGCGAAAGGCCGAT | |
| | TCACCATCTCCAAGACCTCGTCGACCACGGTGACCCTGCAAATGACCAGTCTG | |
| | ACAGCCGCGGACACGGCCACCTATTTCTGTGCGAGAGATCTGGGTGCTGCTGG | |
| | TGATGCTTATAACTTGTGGGGGCCAGGCACCCTGGTCACCGTCTCCTCAGGGC | |
| | AACCTAAGGCTCCATCAGTCTTCCCACTGGCCCCCTGCTGCGGGGACACACCC | |
| | AGCTCCACGGTGACCCTGGGCTGCCTGGTCAAAGGCTACCTCCCGGAGCCAGT | |
| | GACCGTGACCTGGAACTCGGGCACCCTCACCAATGGGGTACGCACCTTCCCGT | |
| | CCGTCCGGCAGTCCTCAGGCCTCTACTCGCTGAGCAGCGTGGTGAGCGTGACC | |
| | TCAAGCAGCCAGCCCGTCACCTGCAACGTGGCCCACCCAGCCACCAACACCAA | |
| | AGTGGACAAGACCGTTGCGCCCTCGACATGCAGCAAGCCCATGTGCCCACCCC | |
| | CTGAACTCCCGGGGGGACCGTCTGTCTTCATCTTCCCCCCAAAACCCAAGGAC | |
| | ACCCTCATGATCTCACGCACCCCCGAGGTCACATGCGTGGTGGTGGACGTGAG | |
| | CCAGGATGACCCCGAGGTGCAGTTCACATGGTACATAAACAACGAGCAGGTGC | |
| | GCACCGCCCGGCCGCCGCTACGGGAGCAGCAGTTCAACAGCACGATCCGCGTG | |
| | GTCAGCACCCTCCCCATCGCGCACCAGGACTGGCTGAGGGGCAAGGAGTTCAA | |
| | GTGCAAAGTCCACAACAAGGCACTCCCGGCCCCCATCGAGAAAACCATCTCCA | |
| | AAGCCAGAGGGCAGCCCCTGGAGCCGAAGGTCTACACCATGGGCCCTCCCCGG | |
| | GAGGAGCTGAGCAGCAGGTCGGTCAGCCTGACCTGCATGATCAACGGCTTCTA | |
| | CCCTTCCGACATCTCGGTGGAGTGGGAGAAGAACGGGAAGGCAGAGGACAACT | |
| | ACAAGACCACGCCGACCGTGCTGGACAGCGACGGCTCCTACTTCCTCTACAGC | |
| | AAGCTCTCAGTGCCCACGAGTGAGTGGCAGCGGGGCGACGTCTTCACCTGCTC | |
| | CGTGATGCACGAGGCCTTGCACAACCACTACACGCAGAAGTCCATCTCCCGCT | |
| | CTCCGGGTAAATAG | |
| 9E6 H | ATGGAGACTGGGCTGCGCTGGCTTCTCCTGGTCGCTGTGCTCAAAGGTGTCCA | 2 |
| | GTGTCAGTCGTTGGAGGAGTCCGGGGGAGACCTGGTCAAGCCTGGGGCATCCC | |
| | TGACACTCACCTGCAAAGCCTCTGGATTCGACTTCAGTAGCAGCTACTACATG | |
| | TGCTGGGTCCGCCAGGCTCCAGGGAGACTGGAGTGGATCGCATGCATTTATGG | |
| | TGGTGGTCTGAGTAACACTTACTACGCGGGCTGGGCAAAAGGCCGATTCACCA | |
| | TCTCCAAAACCTCGTCGACCACGGTGACTCTGCAAATGACCAGTCTGACAGTC | |
| | GCGGACACGGCCACCTATTTCTGTGCGAGAGATGCTGGGACTAGTGGTGATTA | |
| | CCTTAACTTGTGGGGCCCGGGCACCCTGGTCACCGTCTCCTCAGGGCAACCTA | |
| | AGGCTCCATCAGTCTTCCCACTGGCCCCCTGCTGCGGGGACACACCCAGCTCC | |
| | ACGGTGACCCTGGGCTGCCTGGTCAAAGGCTACCTCCCGGAGCCAGTGACCGT | |
| | GACCTGGAACTCGGGCACCCTCACCAATGGGGTACGCACCTTCCCGTCCGTCC | |
| | GGCAGTCCTCAGGCCTCTACTCGCTGAGCAGCGTGGTGAGCGTGACCTCAAGC | |
| | AGCCAGCCCGTCACCTGCAACGTGGCCCACCCAGCCACCAACACCAAAGTGGA | |
| | CAAGACCGTTGCGCCCTCGACATGCAGCAAGCCCATGTGCCCACCCCCTGAAC | |
| | TCCCGGGGGGACCGTCTGTCTTCATCTTCCCCCCAAAACCCAAGGACACCCTC | |
| | ATGATCTCACGCACCCCCGAGGTCACATGCGTGGTGGTGGACGTGAGCCAGGA | |
| | TGACCCCGAGGTGCAGTTCACATGGTACATAAACAACGAGCAGGTGCGCACCG | |
| | CCCGGCCGCCGCTACGGGAGCAGCAGTTCAACAGCACGATCCGCGTGGTCAGC | |
| | ACCCTCCCCATCGCGCACCAGGACTGGCTGAGGGGCAAGGAGTTCAAGTGCAA | |
| | AGTCCACAACAAGGCACTCCCGGCCCCCATCGAGAAAACCATCTCCAAAGCCA | |
| | GAGGGCAGCCCCTGGAGCCGAAGGTCTACACCATGGGCCCTCCCCGGGAGGAG | |
| | CTGAGCAGCAGGTCGGTCAGCCTGACCTGCATGATCAACGGCTTCTACCCTTC | |
| | CGACATCTCGGTGGAGTGGGAGAAGAACGGGAAGGCAGAGGACAACTACAAGA | |
| | CCACGCCGACCGTGCTGGACAGCGACGGCTCCTACTTCCTCTACAGCAAGCTC | |
| | TCAGTGCCCACGAGTGAGTGGCAGCGGGGCGACGTCTTCACCTGCTCCGTGAT | |
| | GCACGAGGCCTTGCACAACCACTACACGCAGAAGTCCATCTCCCGCTCTCCGG | |
| | GTAAATAG | |
| 11F9 H | ATGGAGACTGGGCTGCGCTGGCTTCTCCTGGTCGCTGTGCTCAAAGGTGTCCA | 3 |
| | GTGTCAGCAGCAGCTGGAGGAGTCCGGGGGAGGCCTGGTCCAGCCTGAGGGAT | |
| | CCCTGACACTCACCTGCATAGCTTCTGGATTCTCCTTCAGTAGCAGCCACTGG | |
| | ATATGTTGGGTCCGCCAGGCTCCAGGGAAGGGGCTGGAGTGGATCGCATGCAT | |
| | GTCTACTAGTAGTGGTAGCACTTACGATGCGAACTGGGCGAAAGGCCGATTCA | |
| | CCATCTCCAAAACCTCGTCGACCACGGTGACTCTGCAAATGACCAGTCTGACA | |
| | GCCGCGGACACGGCCACCTATTTCTGTGCGAGAGATGTTGGCGGTAGTACTAC | |
| | TTACTTTGACTTGTGGGGCCCAGGCACCCTGGTCACCGTCTCCTCAGGGCAAC | |
| | CTAAGGCTCCATCAGTCTTCCCACTGGCCCCCTGCTGCGGGGACACACCCAGC | |
| | TCCACGGTGACCCTGGGCTGCCTGGTCAAAGGCTACCTCCCGGAGCCAGTGAC | |
| | CGTGACCTGGAACTCGGGCACCCTCACCAATGGGGTACGCACCTTCCCGTCCG | |
| | TCCGGCAGTCCTCAGGCCTCTACTCGCTGAGCAGCGTGGTGAGCGTGACCTCA | |
| | AGCAGCCAGCCCGTCACCTGCAACGTGGCCCACCCAGCCACCAACACCAAAGT | |
| | GGACAAGACCGTTGCGCCCTCGACATGCAGCAAGCCCATGTGCCCACCCCCTG | |
| | AACTCCCGGGGGGACCGTCTGTCTTCATCTTCCCCCCAAAACCCAAGGACACC | |
| | CTCATGATCTCACGCACCCCCGAGGTCACATGCGTGGTGGTGGACGTGAGCCA | |
| | GGATGACCCCGAGGTGCAGTTCACATGGTACATAAACAACGAGCAGGTGCGCA | |
| | CCGCCCGGCCGCCGCTACGGGAGCAGCAGTTCAACAGCACGATCCGCGTGGTC | |
| | AGCACCCTCCCCATCGCGCACCAGGACTGGCTGAGGGGCAAGGAGTTCAAGTG | |
| | CAAAGTCCACAACAAGGCACTCCCGGCCCCCATCGAGAAAACCATCTCCAAAG | |
| | CCAGAGGGCAGCCCCTGGAGCCGAAGGTCTACACCATGGGCCCTCCCCGGGAG | |
| | GAGCTGAGCAGCAGGTCGGTCAGCCTGACCTGCATGATCAACGGCTTCTACCC | |
| | TTCCGACATCTCGGTGGAGTGGGAGAAGAACGGGAAGGCAGAGGACAACTACA | |
| | AGACCACGCCGACCGTGCTGGACAGCGACGGCTCCTACTTCCTCTACAGCAAG | |
| | CTCTCAGTGCCCACGAGTGAGTGGCAGCGGGGCGACGTCTTCACCTGCTCCGT | |
| | GATGCACGAGGCCTTGCACAACCACTACACGCAGAAGTCCATCTCCCGCTCTC | |
| | CGGGTAAATAG | |
| 11G5 H | ATGGAGACTGGGCTGCGCTGGCTTCTCCTGGTCGCTGTGCTCAAAGGTGTCCA | 4 |
| | GTGTCAGTCGTTGGAGGAGTCCGGGGGAGACCTGGTCAAGCCTGGGGCATCCC | |
| | TGACACTCACCTGCACAGCTTCTGGATTCTCCTTCAGTAGCAGCTACTACATG | |
| | TGCTGGGTCCGCCAGGCTCCAGGGAAGAGGCTGGAGTGGATCGCTTGCATTTA | |
| | TGGTGGTGGTCTGAGTAACACTTACTACGCGGGCTGGGCAAAAGGCCGATTCA | |
| | CCATCTCCAAAACCTCGTCGACCACGGTGACTCTGCAAATGACCAGTCTGACA | |
| | GCCGCGGACACGGCCACCTATTTCTGTGCGAGAGATGCTGGGACTAGTGGTGA | |
| | TTACCTTAACTTGTGGGGCCCAGGCACCCTGGTCACCGTCTCCTCAGGGCAAC | |
| | CTAAGGCTCCATCAGTCTTCCCACTGGCCCCCTGCTGCGGGGACACACCCAGC | |
| | TCCACGGTGACCCTGGGCTGCCTGGTCAAAGGCTACCTCCCGGAGCCAGTGAC | |
| | CGTGACCTGGAACTCGGGCACCCTCACCAATGGGGTACGCACCTTCCCGTCCG | |
| | TCCGGCAGTCCTCAGGCCTCTACTCGCTGAGCAGCGTGGTGAGCGTGACCTCA | |
| | AGCAGCCAGCCCGTCACCTGCAACGTGGCCCACCCAGCCACCAACACCAAAGT | |
| | GGACAAGACCGTTGCGCCCTCGACATGCAGCAAGCCCATGTGCCCACCCCCTG | |
| | AACTCCCGGGGGGACCGTCTGTCTTCATCTTCCCCCCAAAACCCAAGGACACC | |
| | CTCATGATCTCACGCACCCCCGAGGTCACATGCGTGGTGGTGGACGTGAGCCA | |
| | GGATGACCCCGAGGTGCAGTTCACATGGTACATAAACAACGAGCAGGTGCGCA | |
| | CCGCCCGGCCGCCGCTACGGGAGCAGCAGTTCAACAGCACGATCCGCGTGGTC | |
| | AGCACCCTCCCCATCGCGCACCAGGACTGGCTGAGGGGCAAGGAGTTCAAGTG | |
| | CAAAGTCCACAACAAGGCACTCCCGGCCCCCATCGAGAAAACCATCTCCAAAG | |
| | CCAGAGGGCAGCCCCTGGAGCCGAAGGTCTACACCATGGGCCCTCCCCGGGAG | |
| | GAGCTGAGCAGCAGGTCGGTCAGCCTGACCTGCATGATCAACGGCTTCTACCC | |
| | TTCCGACATCTCGGTGGAGTGGGAGAAGAACGGGAAGGCAGAGGACAACTACA | |
| | AGACCACGCCGACCGTGCTGGACAGCGACGGCTCCTACTTCCTCTACAGCAAG | |
| | CTCTCAGTGCCCACGAGTGAGTGGCAGCGGGGCGACGTCTTCACCTGCTCCGT | |
| | GATGCACGAGGCCTTGCACAACCACTACACGCAGAAGTCCATCTCCCGCTCTC | |
| | CGGGTAAATAG | |
| 16F5 H | ATGGAGACTGGGCTGCGCTGGCTTCTCCTGGTCGCTGTGCTCAAAGGTGTCCA | 5 |
| | GTGTCAGCAGCAGTTGGAGGAGTCCGGGGGAGGCCTGGTCAAGCCTGGAGGAA | |
| | CCCTGACACTCACCTGCAAAGCCTCTGGAATCGACTTCAGTAACTACTACTAC | |
| | ATGTGCTGGGTCCGCCAGGCTCCAGGGAAGGGGCTGGAGTTGATCGCATGCAT | |
| | TTATACTGGTAGTAGTGGTAGCACATGGTACGCGACCTGGGCGAAGGGCCGAT | |
| | TCACCATCTCCAAAACCTCGTCGACCACGGTGACTCTGCAAATGACCAGTCTG | |
| | ACAGCCGCGGACACGGCCACCTATTTCTGTGCGAGAGATCGTGATGTTGGTAG | |
| | TCTTTATGACTCCTTAGATCTCTGGGGCCAGGGCACCCTGGTCACCGTCTCTC | |
| | CAGGGCAACCTAAGGCTCCATCAGTCTTCCCACTGGCCCCCTGCTGCGGGGAC | |
| | ACACCCAGCTCCACGGTGACCCTGGGCTGCCTGGTCAAAGGCTACCTCCCGGA | |
| | GCCAGTGACCGTGACCTGGAACTCGGGCACCCTCACCAATGGGGTACGCACCT | |
| | TCCCGTCCGTCCGGCAGTCCTCAGGCCTCTACTCGCTGAGCAGCGTGGTGAGC | |
| | GTGACCTCAAGCAGCCAGCCCGTCACCTGCAACGTGGCCCACCCAGCCACCAA | |
| | CACCAAAGTGGACAAGACCGTTGCGCCCTCGACATGCAGCAAGCCCATGTGCC | |
| | CACCCCCTGAACTCCCGGGGGGACCGTCTGTCTTCATCTTCCCCCCAAAACCC | |
| | AAGGACACCCTCATGATCTCACGCACCCCCGAGGTCACATGCGTGGTGGTGGA | |
| | CGTGAGCCAGGATGACCCCGAGGTGCAGTTCACATGGTACATAAACAACGAGC | |
| | AGGTGCGCACCGCCCGGCCGCCGCTACGGGAGCAGCAGTTCAACAGCACGATC | |
| | CGCGTGGTCAGCACCCTCCCCATCGCGCACCAGGACTGGCTGAGGGGCAAGGA | |
| | GTTCAAGTGCAAAGTCCACAACAAGGCACTCCCGGCCCCCATCGAGAAAACCA | |
| | TCTCCAAAGCCAGAGGGCAGCCCCTGGAGCCGAAGGTCTACACCATGGGCCCT | |
| | CCCCGGGAGGAGCTGAGCAGCAGGTCGGTCAGCCTGACCTGCATGATCAACGG | |
| | CTTCTACCCTTCCGACATCTCGGTGGAGTGGGAGAAGAACGGGAAGGCAGAGG | |
| | ACAACTACAAGACCACGCCGACCGTGCTGGACAGCGACGGCTCCTACTTCCTC | |
| | TACAGCAAGCTCTCAGTGCCCACGAGTGAGTGGCAGCGGGGCGACGTCTTCAC | |
| | CTGCTCCGTGATGCACGAGGCCTTGCACAACCACTACACGCAGAAGTCCATCT | |
| | CCCGCTCTCCGGGTAAATAG | |
| 19B1 H | ATGGAGACTGGGCTGCGGTGGCTTCTCCTGGTCGCTGTGCTCAAAGGTGTCCA | 6 |
| | GTGTCAGGAGCAGCTGGTGGAGTCCGGGGGAGGCCTGGTCCAGCCTGAGGGAT | |
| | CCCTGACACTCACCTGCACAGCTTCTGGATTCTCCTTCAGTGACAGCTACTAC | |
| | ATGTGCTGGGTCCGCCAGGCTCCAGGGAAGGGGCTGGAGTGGATCGCATGCAT | |
| | TTATGGTGGTACTATTACTAATACTTACTACGCGAGCTGGGCGAAAGGCCGAT | |
| | TCACCATCTCCAAGACCTCGTCGACCACGGTGACCCTGCAAATGACCAGTCTG | |
| | ACAGCCGCGGACACGGCCACCTATTTCTGTGCGAGAGATCTGGGTGCTGCTGG | |
| | TGATGCTTATAACTTGTGGGGCCCAGGCACCCTGGTCACCGTCTCCTCAGGGC | |
| | AACCTAAGGCTCCATCAGTCTTCCCACTGGCCCCCTGCTGCGGGGACACACCC | |
| | AGCTCCACGGTGACCCTGGGCTGCCTGGTCAAAGGCTACCTCCCGGAGCCAGT | |
| | GACCGTGACCTGGAACTCGGGCACCCTCACCAATGGGGTACGCACCTTCCCGT | |
| | CCGTCCGGCAGTCCTCAGGCCTCTACTCGCTGAGCAGCGTGGTGAGCGTGACC | |
| | TCAAGCAGCCAGCCCGTCACCTGCAACGTGGCCCACCCAGCCACCAACACCAA | |
| | AGTGGACAAGACCGTTGCGCCCTCGACATGCAGCAAGCCCATGTGCCCACCCC | |
| | CTGAACTCCCGGGGGGACCGTCTGTCTTCATCTTCCCCCCAAAACCCAAGGAC | |
| | ACCCTCATGATCTCACGCACCCCCGAGGTCACATGCGTGGTGGTGGACGTGAG | |
| | CCAGGATGACCCCGAGGTGCAGTTCACATGGTACATAAACAACGAGCAGGTGC | |
| | GCACCGCCCGGCCGCCGCTACGGGAGCAGCAGTTCAACAGCACGATCCGCGTG | |
| | GTCAGCACCCTCCCCATCGCGCACCAGGACTGGCTGAGGGGCAAGGAGTTCAA | |
| | GTGCAAAGTCCACAACAAGGCACTCCCGGCCCCCATCGAGAAAACCATCTCCA | |
| | AAGCCAGAGGGCAGCCCCTGGAGCCGAAGGTCTACACCATGGGCCCTCCCCGG | |
| | GAGGAGCTGAGCAGCAGGTCGGTCAGCCTGACCTGCATGATCAACGGCTTCTA | |
| | CCCTTCCGACATCTCGGTGGAGTGGGAGAAGAACGGGAAGGCAGAGGACAACT | |
| | ACAAGACCACGCCGACCGTGCTGGACAGCGACGGCTCCTACTTCCTCTACAGC | |
| | AAGCTCTCAGTGCCCACGAGTGAGTGGCAGCGGGGCGACGTCTTCACCTGCTC | |
| | CGTGATGCACGAGGCCTTGCACAACCACTACACGCAGAAGTCCATCTCCCGCT | |
| | CTCCGGGTAAATAG | |
| 2C5 L | ATGGACACGAGGGCCCCCACTCAGCTGCTGGGGCTCCTGCTGCTCTGGCTCCC | 7 |
| | AGGTGCCAGATGTGCCTCTGATATGACCCAGACTCCAGCCTCCGTGTCTGCAG | |
| | CTGTGGGAGGCACAGTCACCATCAAGTGCCAGGCCAGTGAGAGCATTTACAGT | |
| | GGTTTGGCCTGGTATCAGCAGAAACCAGGGCAGCCTCCCAAGCTCCTGATCTT | |
| | TGATGCATCCGATCTGGCATCTGGGGTCCCATCGCGGTTCAAAGGCAGTAGAT | |
| | CTGAGACAGAGTACACTCTCACCATCAGCGACCTGGAGTGTGCCGATGCTGCC | |
| | ACTTACTACTGTCAATGCACTGATCGTAATAGTATTACTTCTTATGCTTTCGG | |
| | CGGAGGGACCGAGGTGGTGGTCAAAGGTGATCCAGTTGCACCTACTGTCCTCA | |
| | TCTTCCCACCAGCTGCTGATCAGGTGGCAACTGGAACAGTCACCATCGTGTGT | |
| | GTGGCGAATAAATACTTTCCCGATGTCACCGTCACCTGGGAGGTGGATGGCAC | |
| | CACCCAAACAACTGGCATCGAGAACAGTAAAACACCGCAGAATTCTGCAGATT | |
| | GTACCTACAACCTCAGCAGCACTCTGACACTGACCAGCACACAGTACAACAGC | |
| | CACAAAGAGTACACCTGCAAGGTGACCCAGGGCACGACCTCAGTCGTCCAGAG | |
| | CTTCAATAGGGGTGACTGTTAG | |
| 9E6 L | ATGGACACGAGGGCCCCCACTCAGCTGCTGGGGCTCCTGCTGCTCTGGCTCCC | 8 |
| | AGGTGCCAGATGTGCCGTCGTGATGACCCAGACTGCATCCCCCGTGTCTGGAG | |
| | CTGTGGGAGGCACAGTCACCATCAATTGCCAGGCCAGTCAGAGTATTAGTAGT | |
| | AGCTACTTATCCTGGTATCAGCAGAAACCAGGGCAGCCTCCCAAGCTCCTGAT | |
| | CTATGGTGCATCCACTCTGGCATCTGGGGTCCCATCGCGGTTCAAAGGCAGTG | |
| | GATCTGGGACACAGTTCACTCTCACCATCAGTGGCGTGCAGTGTGACGATGCT | |
| | GCCACTTACTACTGTGCATACGATGCTTATCGTCTCAGTAGTCCTGATAATAT | |
| | TTTCGGCGGAGGGACCGAGGTGGTGGTCAAAGGTGATCCAGTTGCACCTACTG | |
| | TCCTCATCTTCCCACCAGCTGCTGATCAGGTGGCAACTGGAACAGTCACCATC | |
| | GTGTGTGTGGCGAATAAATACTTTCCCGATGTCACCGTCACCTGGGAGGTGGA | |
| | TGGCACCACCCAAACAACTGGCATCGAGAACAGTAAAACACCGCAGAATTCTG | |
| | CAGATTGTACCTACAACCTCAGCAGCACTCTGACACTGACCAGCACACAGTAC | |
| | AACAGCCACAAAGAGTACACCTGCAAGGTGACCCAGGGCACGACCTCAGTCGT | |
| | CCAGAGCTTCAATAGGGGTGACTGTTAG | |
| 11F9 L | ATGGACACGAGGGCCCCCACTCAGCTGCTGGGGCTCCTGCTGCTCTGGCTCCC | 9 |
| | AGGTGCCAGATGTGCCTATGATATGACCCAGACTCCAGCCTCCGTGTCTGAAC | |
| | CTGTGGGAGGCACAGTCACCATCAAGTGCCAGGCCAGTCAGAGCATTAGCAAT | |
| | GAATTATCCTGGTATCAACAAAAACCAGGGCAGCCTCCCAAACTCCTGATCTA | |
| | CAGGGCATCCACTCTGGCATCTGGGGTCCCATCGCGGTTCAAAGGCAGTGGAT | |
| | CTGGGACACAGTTCACTCTCACCATCAACGGCGTGGAGTGTGCCGATGCTGCC | |
| | ACTTACTACTGTCAACAGGGTTATAGTATTAGTAATGTTGATAATACTTTCGG | |
| | CGGAGGGACCGAGGTGGTGGTCAAAGGTGATCCAGTTGCACCTACTGTCCTCA | |
| | TCTTCCCACCAGCTGCTGATCAGGTGGCAACTGGAACAGTCACCATCGTGTGT | |
| | GTGGCGAATAAATACTTTCCCGATGTCACCGTCACCTGGGAGGTGGATGGCAC | |
| | CACCCAAACAACTGGCATCGAGAACAGTAAAACACCGCAGAATTCTGCAGATT | |
| | GTACCTACAACCTCAGCAGCACTCTGACACTGACCAGCACACAGTACAACAGC | |
| | CACAAAGAGTACACCTGCAAGGTGACCCAGGGCACGACCTCAGTCGTCCAGAG | |
| | CTTCAATAGGGGTGACTGTTAG | |
| 11G5 L | ATGGACACGAGGGCCCCCACTCAGCTGCTGGGGCTCCTGCTGCTCTGGCTCCC | 10 |
| | AGGTGCCAGATGTGCCGTCGTGATGACCCAGACTGCATCCCCCGTGTCTGGAG | |
| | CTGTGGGAGGCACAGTCACCATCAATTGCCAGGCCAGTCAGAGTATTAGTGCT | |
| | AGCGCCTTATCCTGGTATCAACAGAAACCAGGGCAGCCTCCCAAGCTCCTGAT | |
| | CTATGCTGCATCCACTCTGGAATCTGGGGTCCCATCGCGGTTCAAAGGCAGTG | |
| | GATCTGGGACACAGTTCACTCTCACCATCAGCGGCGTGCAGTGTGACGATGCT | |
| | GCCACTTACTACTGTGCATACGATGGTTATCGTCTCAGTAGTGCTGATAATAT | |
| | TTTCGGCGGAGGGACCGAGGTGGTGGTCAAAGGTGATCCAGTTGCACCTACTG | |
| | TCCTCATCTTCCCACCAGCTGCTGATCAGGTGGCAACTGGAACAGTCACCATC | |
| | GTGTGTGTGGCGAATAAATACTTTCCCGATGTCACCGTCACCTGGGAGGTGGA | |
| | TGGCACCACCCAAACAACTGGCATCGAGAACAGTAAAACACCGCAGAATTCTG | |
| | CAGATTGTACCTACAACCTCAGCAGCACTCTGACACTGACCAGCACACAGTAC | |
| | AACAGCCACAAAGAGTACACCTGCAAGGTGACCCAGGGCACGACCTCAGTCGT | |
| | CCAGAGCTTCAATAGGGGTGACTGTTAG | |
| 16F5 L | ATGGACACGAGGGCCCCCACTCAGCTGCTGGGGCTCCTGCTGCTCTGGCTCCC | 11 |
| | AGGTGCCAGATGTGCCTATGATATGACCCAGACTCCAGCCTCCGTGGAGGCAG | |
| | CTGTGGGAGGCACAGTCACCATCAATTGCCAGGCCAGTCAGAGCATTAATAAT | |
| | TGGTTATCCTGGTATCAGCAGAAACCAGGGCAGCGTCCCAAGCTCCTGATTTA | |
| | CCAGGCATCCACTCTGGCATCTGGGGTCTCATCGCGGTTCAAAGGCAGTGGAT | |
| | CTGGGACACACTTTACTCTCACCATCAGCGACCTGGAGTGTGCCGATGCTGCC | |
| | ACTTACTACTGTCAACAGGGTTGGAGTATAGACGATATTGATAATGCTTTCGG | |
| | CGGAGGGACCGAGGTGGTGGTCAAGGGTGATCCAGTTGCACCTACTGTCCTCA | |
| | TCTTCCCACCAGCTGCTGATCAGGTGGCAACTGGAACAGTCACCATCGTGTGT | |
| | GTGGCGAATAAATACTTTCCCGATGTCACCGTCACCTGGGAGGTGGATGGCAC | |
| | CACCCAAACAACTGGCATCGAGAACAGTAAAACACCGCAGAATTCTGCAGATT | |
| | GTACCTACAACCTCAGCAGCACTCTGACACTGACCAGCACACAGTACAACAGC | |
| | CACAAAGAGTACACCTGCAAGGTGACCCAGGGCACGACCTCAGTCGTCCAGAG | |
| | CTTCAATAGGGGTGACTGTTAG | |
| 19B1 L | ATGGACACGAGGGCCCCCACTCAGCTGCTGGGGCTCCTGCTGCTCTGGCTCCC | 12 |
| | AGGTGCCAGATGTGCCTCTGATATGACCCAGACTCCAGCCTCCGTGTCTGAAC | |
| | CTGTGGGAGGCACAGTCACCATCAAGTGCCAGGCCAGTGAGAACATTTACAGC | |
| | TCTTTAGCCTGGTATCAGCAGAAACCAGGGCAGCCTCCCAAGCTCCTGATCTA | |
| | TGATGCATCCAATCTGGCATCTGGGGTCCCATCGCGGTTCAGCGGCAGTGGAT | |
| | CTGGGACAGAGTTCACTCTCACCATCAGCGACCTGGAGTGTGCCGATGCTGCC | |
| | ACTTACTACTGTCAATGCACTTATCGTAGTAGTAGTAGTTCTTATGCTTTCGG | |
| | CGGAGGGACCGAGGTGGTGGTCAAAGGTGATCCAGTTGCACCTACTGTCCTCA | |
| | TCTTCCCACCAGCTGCTGATCAGGTGGCAACTGGAACAGTCACCATCGTGTGT | |
| | GTGGCGAATAAATACTTTCCCGATGTCACCGTCACCTGGGAGGTGGATGGCAC | |
| | CACCCAAACAACTGGCATCGAGAACAGTAAAACACCGCAGAATTCTGCAGATT | |
| | GTACCTACAACCTCAGCAGCACTCTGACACTGACCAGCACACAGTACAACAGC | |
| | CACAAAGAGTACACCTGCAAGGTGACCCAGGGCACGACCTCAGTCGTCCAGAG | |
| | CTTCAATAGGGGTGACTGTTAG | |

Table 4 shows the deduced amino acid sequences of the variable domains of antibodies 2C5, 9E6, 11F9, 11G5, 16F5, and 19B1. The complementarity-determining regions (CDRs) are in boldface and underlined.

TABLE 4
Variable domain amino acid sequences of exemplary antibodies

| Ab Chain | Sequence (N-terminus to C-terminus) | SEQ |
|---|---|---|
| 2C5 VH | QEQLVESGGD LVKPGASLTL TCTASGFSFS SGYYMCWVRQ APGKGLEWIA | 13 |
| | CIYGGALTNT YYATWAKGRF TISKTSSTTV TLQMTSLTAA DTATYFCARD | |
| | LGAAGDAYNL WGPGTLVTVS S | |
| 9E6 VH | QSLEESGGDL VKPGASLTLT CKASGFDFSS SYYMCWVRQA PGRLEWIACI | 14 |
| | YGGGLSNTYY AGWAKGRFTI SKTSSTTVTL QMTSLTVADT ATYFCARDAG | |
| | TSGDYLNLWG PGTLVTVSS | |
| 11F9 VH | QQQLEESGGG LVQPEGSLTL TCIASGFSFS SSHWICWVRQ APGKGLEWIA | 15 |
| | CMSTSSGSTY DANWAKGRFT ISKTSSTTVT LQMTSLTAAD TATYFCARDV | |
| | GGSTTYFDLW GPGTLVTVSS | |
| 11G5 VH | QSLEESGGDL VKPGASLTLT CTASGFSFSS SYYMCWVRQA PGKRLEWIAC | 16 |
| | IYGGGLSNTY YAGWAKGRFT ISKTSSTTVT LQMTSLTAAD TATYFCARDA | |
| | TSGDYLNLWG PGTLVTVSS | |
| 16F5 VH | QQQLEESGGG LVKPGGTLTL TCKASGIDFS NYYYMCWVRQ APGKGLELIA | 17 |
| | CIYTGSSGST WYATWAKGRF TISKTSSTTV TLQMTSLTAA DTATYFCARD | |
| | RDVGSLYDSL DLWGQGTLVT VSP | |
| 19B1 VH | QEQLVESGGG LVQPEGSLTL TCTASGFSFS DSYYMCWVRQ APGKGLEWIA | 18 |
| | CIYGGTITNT YYASWAKGRF TISKTSSTTV TLQMTSLTAA DTATYFCARD | |
| | LGAAGDAYNL WGPGTLVTVS S | |
| 2C5 VL | ASDMTQTPAS VSAAVGGTVT IKCQASESIY SGLAWYQQKP GQPPKLLIFD | 19 |
| | ASDLASGVPS RFKGSRSETE YTLTISDLEC ADAATYYCQC TDRNSITSYA | |
| | FGGGTEVVVK | |
| 9E6 VL | AVVMTQTASP VSGAVGGTVT INCQASQSIS SSYLSWYQQK PGQPPKLLIY | 20 |
| | GASTLASGVP SRFKGSGSGT QFTLTISGVQ CDDAATYYCA YDAYRLSSPD | |
| | NIFGGGTEVV VK | |
| 11F9 VL | AYDMTQTPAS VSEPVGGTVT IKCQASQSIS NELSWYQQKP GQPPKLLIYR | 21 |
| | ASTLASGVPS RFKGSGSGTQ FTLTINGVEC ADAATYYCQQ GYSISNVDNT | |
| | FGGGTEVVVK | |
| 11G5 VL | AVVMTQTASP VSGAVGGTVT INCQASQSIS ASALSWYQQK PGQPPKLLIY | 22 |
| | AASTLESGVP SRFKGSGSGT QFTLTISGVQ CDDAATYYCA YDGYRLSSAD | |
| | NIFGGGTEVV VK | |
| 16F5 VL | AYDMTQTPAS VEAAVGGTVT INCQASQSIN NWLSWYQQKP GQRPKLLIYQ | 23 |
| | ASTLASGVSS RFKGSGSGTH FTLTISDLEC ADAATYYCQQ GWSIDDIDNA | |
| | FGGGTEVVVK | |
| 19B1 VL | ASDMTQTPAS VSEPVGGTVT IKCQASENIY SSLAWYQQKP GQPPKLLIYD | 24 |
| | ASNLASGVPS RFSGSGSGTE FTLTISDLEC ADAATYYCQC TYRSSSSSYA | |
| | FGGGTEVVVK | |

Table 5 shows heavy and light chain constant region amino acid sequences (CH and CL, respectively) of the six antibodies 2C5, 9E6, 11F9, 11G5, 16F5, and 19B1.

TABLE 5
Constant region amino acid sequences of exemplary antibodies

| Ab Chain | Sequence (N-terminal to C-terminal) | SEQ |
|---|---|---|
| CH | GQPKAPSVFP LAPCCGDTPS STVTLGCLVK GYLPEPVTVT WNSGTLTNGV | 25 |
| | RTFPSVRQSS GLYSLSSVVS VTSSSQPVTC NVAHPATNTK VDKTVAPSTC | |
| | SKPMCPPPEL PGGPSVFIFP PKPKDTLMIS RTPEVTCVVV DVSQDDPEVQ | |
| | FTWYINNEQV RTARPPLREQ QFNSTIRVVS TLPIAHQDWL RGKEFKCKVH | |
| | NKALPAPIEK TISKARGQPL EPKVYTMGPP REELSSRSVS LTCMINGFYP | |
| | SDISVEWEKN GKAEDNYKTT PTVLDSDGSY FLYSKLSVPT SEWQRGDVFT | |
| | CSVMHEALHN HYTQKSISRS PGK | |
| CL | GDPVAPTVLI FPPAADQVAT GTVTIVCVAN KYFPDVTVTW EVDGTTQTTG | 26 |
| | IENSKTPQNS ADCTYNLSST LTLTSTQYNS HKEYTCKVTQ GTTSVVQSFN | |
| | RGDC | |

Table 6 shows heavy CDR (HCDR) and light chain CDR (LCDR) amino acid sequences of antibodies 2C5, 9E6, 11F9, 11G5, 16F5, and 19B1, wherein the CDRs are defined according to the Kabat numbering system. The SEQ ID NOs of the sequences are shown in parentheses.

TABLE 6
CDR amino acid sequences of exemplary antibodies

| | Amino Acid Sequence (N-terminus to C-terminus) | | | | | |
|---|---|---|---|---|---|---|
| Ab | HCDR1 | HCDR2 | HCDR3 | LCDR1 | LCDR2 | LCDR3 |
| 2C5 | FSFSSGYYMC | WIACIYGGAL | ARDLGAAGDA | ESIYSGLA | LLIFDASDLA | QCTDRNSITS |
| | (27) | TNTYYATWA | YN (29) | (30) | S (31) | YA (32) |
| | | (28) | | | | |
| 9E6 | FDFSSSYYMC | WIACIYGGGL | ARDAGTSGDY | QSISSSYL | LLIYGASTLA | AYDAYRLSSP |
| | (33) | SNTYYAGWA | LN (35) | S (36) | S (37) | DNI (38) |
| | | (34) | | | | |
| 11F9 | FSFSSSHWIC | WIACMSTSSG | ARDVGGSTTY | QSISNELS | LLIYRASTLA | QQGYSISNVD |
| | (39) | STYDANWA | FD (41) | (42) | S (43) | NT (44) |
| | | (40) | | | | |
| 11G5 | FSFSSSYYMC | WIACIYGGGL | ARDAGTSGDY | QSISASAL | LLIYAASTLE | AYDGYRLSSA |
| | (45) | SNTYYAGWA | LN (47) | S (48) | S (49) | DNI (50) |
| | | (46) | | | | |
| 16F5 | IDFSNYYYMC | LIACIYTGSS | ARDRDVGSLY | QSINNWLS | LLIYQASTLA | QQGWSIDDID |
| | (51) | GSTWYATWA | DSLD (53) | (54) | S (55) | NA (56) |
| | | (52) | | | | |
| 19B1 | FSFSDSYYMC | WIACIYGGTI | ARDLGAAGDA | ENIYSSLA | LLIYDASNLA | QCTYRSSSSS |
| | (57) | TNTYYASWA | YN (59) | (60) | S (61) | YA (62) |
| | | (58) | | | | |

Table 7 shows SEQ ID NO information for antibodies 2C5, 9E6, 11F9, 11G5, 16F5, and 19B1. Except as indicated by “nt” (nucleotides), all sequences in the table are amino acid sequences.

TABLE 7
SEQ ID NOs for 2C5, 9E6, 11F9, 11G5, 16F5, and 19B1

| | HC | LC | | | | | | | HCDR | | | LCDR | | |
|---|---|---|---|---|---|---|---|---|---|---|---|---|---|---|
| Clone | (nt.) | (nt.) | VH | VL | CH | CL | HC | LC | 1 | 2 | 3 | 1 | 2 | 3 |
| 2C5 | 1 | 7 | 13 | 19 | 25 | 26 | 63 | 69 | 27 | 28 | 29 | 30 | 31 | 32 |
| 9E6 | 2 | 8 | 14 | 20 | 25 | 26 | 64 | 70 | 33 | 34 | 35 | 36 | 37 | 38 |
| 11F9 | 3 | 9 | 15 | 21 | 25 | 26 | 65 | 71 | 39 | 40 | 41 | 42 | 43 | 44 |
| 11G5 | 4 | 10 | 16 | 22 | 25 | 26 | 66 | 72 | 45 | 46 | 47 | 48 | 49 | 50 |
| 16F5 | 5 | 11 | 17 | 23 | 25 | 26 | 67 | 73 | 51 | 52 | 53 | 54 | 55 | 56 |
| 19B1 | 6 | 12 | 18 | 24 | 25 | 26 | 68 | 74 | 57 | 58 | 59 | 60 | 61 | 62 |

### Example 3: Binding Affinity and Epitope of the Rabbit Anti-hIgG Antibodies

The amplified PCR products containing the heavy and light chain antibody coding sequences were cloned into expression vectors and used for transient transfection and antibody purification. Using amine coupling, a mouse anti-rabbit mAb was directly immobilized on CM5 chip (Chu et al., Sci Rep. (2015) 4:7360). The purified rabbit mAb clones were then injected, followed by flow over 1:2 serially diluted hFab, hIgG1, hIgG4 and cynoIgG, ranging from 80-0 nM.

As illustrated in Table 8, all the clones exhibited a binding affinity (KD) greater than sub-nanomolar to the hIgG1, hIgG4 and hFab, but not to cynoIgG. Under the same conditions, the control commercial clone MCA5748G (Bio-Rad, Hercules, CA), however, was able to bind to hIgG1 and hIgG4, but not hFab and cynoIgG.

TABLE 8
Biacore kinetics of binding of rabbit mAb clones to different antibody types

| Sample | Antigen | ka (1/Ms) | kd (1/s) | KD (M) |
|---|---|---|---|---|
| 11F9 | hFab | 2.97E+07 | 3.34E−02 | 1.12E−09 |
| | hIgG4 | 1.45E+05 | 4.06E−05 | 2.79E−10 |
| | hIgG1 | 1.49E+05 | 9.21E−05 | 6.19E−10 |
| | cynoIgG | no binding | | |
| 11G5 | hFab | 8.99E+05 | 5.31E−04 | 5.91E−10 |
| | hIgG4 | 1.35E+05 | 5.80E−06 | 4.29E−11 |
| | hIgG1 | 1.35E+05 | 2.52E−05 | 1.87E−10 |
| | cynoIgG | no binding | | |
| 16F5 | hFab | 3.02E+06 | 5.84E−03 | 1.93E−09 |
| | hIgG4 | 1.57E+05 | 8.09E−05 | 5.14E−10 |
| | hIgG1 | 1.73E+05 | 2.21E−05 | 1.27E−10 |
| | cynoIgG | no binding | | |
| 19B1 | hFab | 1.22E+06 | 6.58E−04 | 5.40E−10 |
| | hIgG4 | 1.36E+05 | 9.14E−06 | 6.70E−11 |
| | hIgG1 | 1.37E+05 | 3.28E−05 | 2.39E−10 |
| | cynoIgG | no binding | | |
| 2C5 | hFab | 2.65E+06 | 1.46E−03 | 5.50E−10 |
| | hIgG4 | 1.35E+05 | 1.36E−05 | 1.00E−10 |
| | hIgG1 | 1.37E+05 | 3.80E−05 | 2.77E−10 |
| | cynoIgG | no binding | | |
| 9E6 | hFab | 1.16E+07 | 6.03E−03 | 5.18E−10 |
| | hIgG4 | 1.37E+05 | 2.68E−05 | 1.96E−10 |
| | hIgG1 | 1.43E+05 | 6.10E−05 | 4.26E−10 |
| | cynoIgG | no binding | | |
| MCA5748G | hFab | no binding | | |
| | hIgG4 | 1.34E+05 | 2.36E−04 | 1.57E−09 |
| | hIgG1 | 1.47E+05 | 2.81E−04 | 1.82E−09 |

| | |
|---|---|
| cynoIgG | no binding |

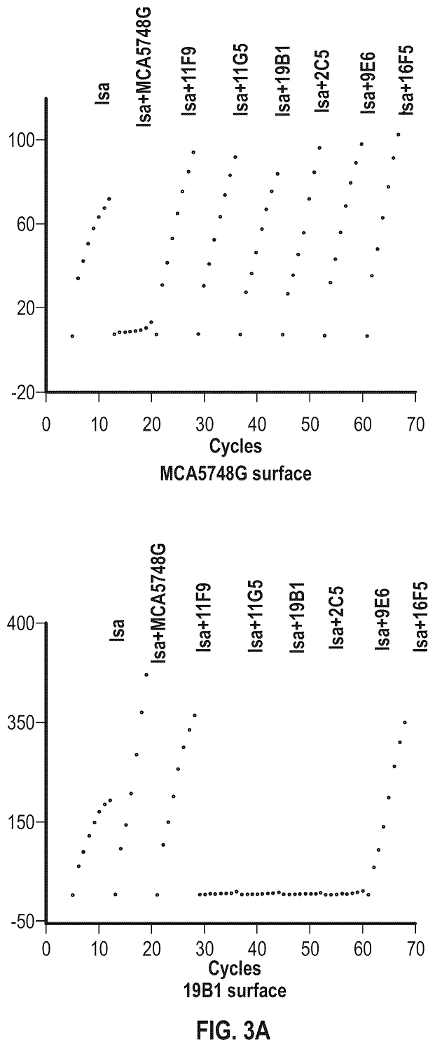

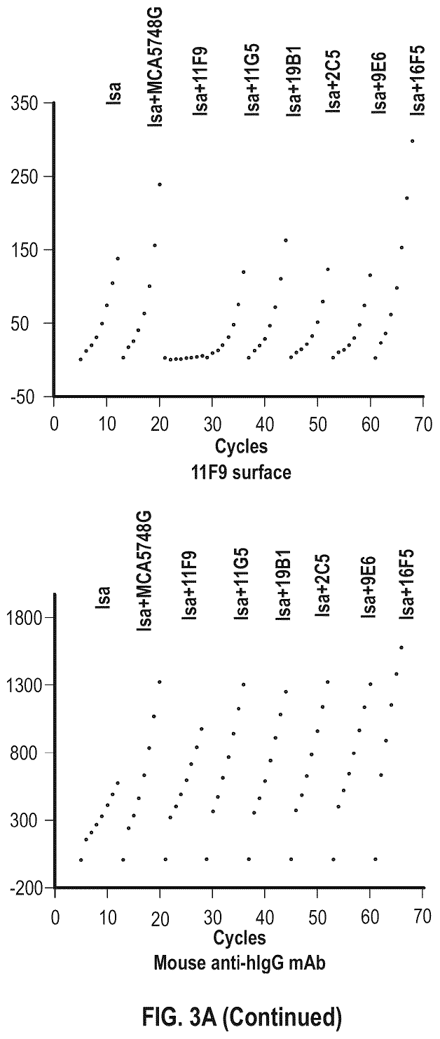

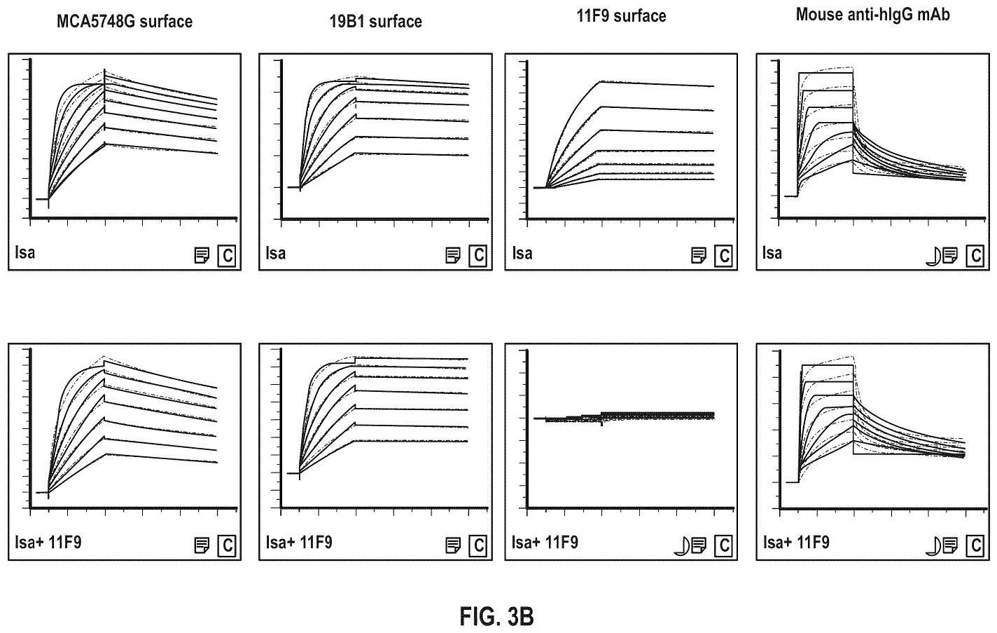

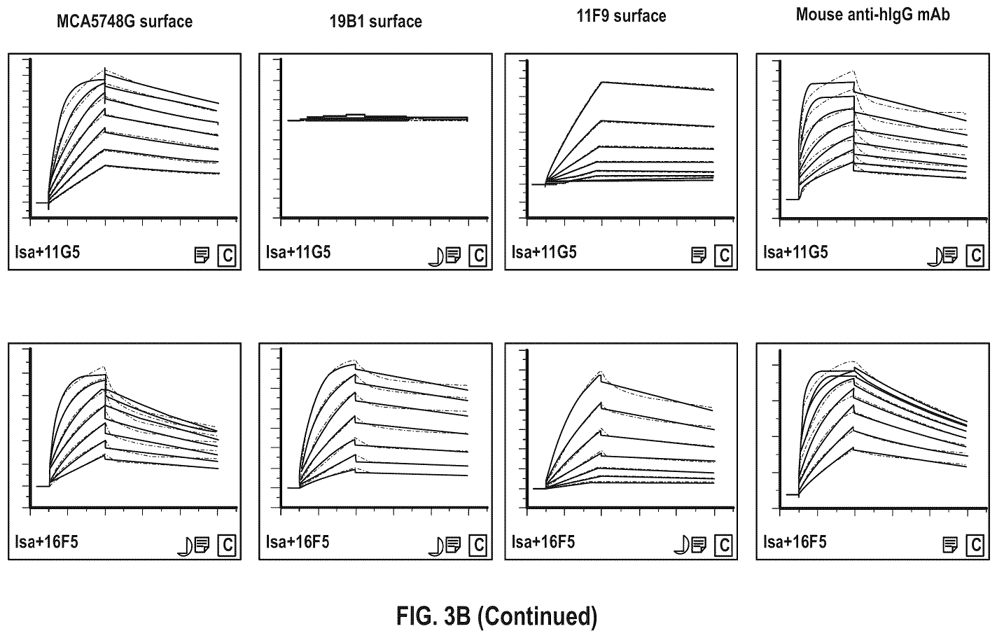

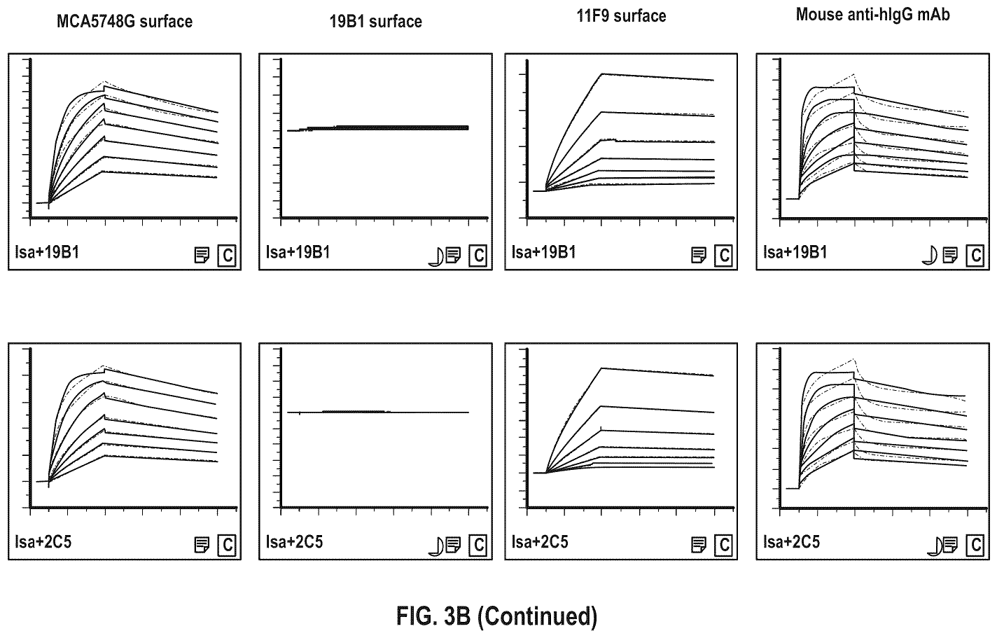

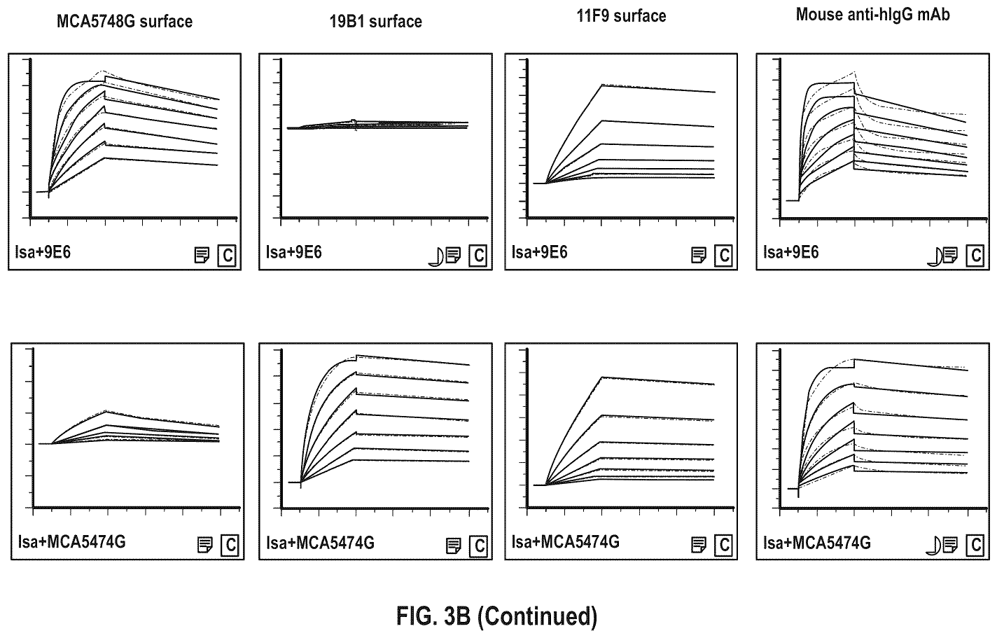

In order to determine if the six rabbit anti-hIgG clones had a different epitope from the only commercially available clone MCA5748G, the MCA5748G, 19B1 and 11F9 clones and a mouse anti-hIgG mAb (as the assay control) were biotinylated. These biotinylated antibodies were then injected into the different flow cells of a streptavidin chip, to reach a range between 500-600 RUs, followed by flow over the 1:2 serially diluted hIgG1 antibody, ranging from 80-0 nM, in the presence of 240 nm of the different rabbit mAb clones as the competitor. As shown in FIGS. 3A and 3B, when MCA5748G was used as the capture antibody on the chip surface, competition was only seen with the clone MAC5748G itself, indicating that this commercial mouse anti-hIgG clone MAC5748G has a different epitope from all the six rabbit anti-hIgG clones disclosed herein.

When the rabbit anti-hIgG clone 19B1 was used as the capture antibody on the chip surface, competition was seen with clones 11G5, 19B1, 2C5 and 9E6. Therefore, the epitope of these four clones are the same, but different from clone 11F9 and 16F5. Furthermore, when clone 11F9 was captured on the chip surface, competition was only seen with clone 11F9 itself, not with clone 16F5, nor with any of the other four clones which share the same epitope. Thus, clone 16F5 also has a different epitope from clone 11F9. These results indicated that clones 16 F5 and 11F9 each has a unique epitope whereas the clones 11G5, 19B1, 2C5 and 9E6 share the same epitope. Paratopes in CDRs determine antibody binding epitopes and the heavy chain plays the dominant role, the epitope classification of the six rabbit anti-hIgG clones correlate well with the phylogenetic tree's relationship (FIG. 2C).

Additional Biacore experiments have shown that all six rabbit anti-hIgG clones are capable of binding to each of the 10 different human IgGs, and the 2 Fabs tested. The ten antibodies included those having human IgG1 and IgG4. Because Fab was used as the immunogen to generate these rabbit anti-hIgG mAb clones, all of them should bind within the Fab region of the IgG. Because different human IgG subtypes (IgG1, IgG2, IgG3 and IgG4) are classified by their Fc sequences, which are not part of the Fab region, all six of the rabbit anti-hIgG antibody clones should be able to recognize all subtypes of human IgGs. This is a significant advantage when these rabbit anti-hIgG mAbs are used for the detection of different subtypes therapeutic human IgG (e.g., IgG1, IgG2, and IgG4) molecules in preclinical studies and/or in process development.

In summary, amino acid sequence alignment showed that all six clones obtained herein were unique and diverse. They bound to three different epitope groups (16F5; 11F9; and 11G5/19B1/2C5/9E6) and demonstrated better binding affinity and targeted different epitopes from the only commercially available mouse-origin anti-hIgG antibody clone, MCA5748G. Clones with different binding epitopes can be used to develop pair-wise human IgG detection assays, such as “sandwich” ELISAs and Gyrolab™ assays.

### Example 4: Specific Detection of Human IgG by Rabbit Antibodies in Gyrolab™ Assay

In order to test if the rabbit anti-hIgG mAb can detect human IgG molecule in the presence of monkey serum, biotinylated clone 16F6 was first used as the capture reagent. Gyrolab™ xPlore, Bioaffy 1000 nL CDs, Rexxip A and Rexxip F Buffer from Gyros Protein Technologies (Uppsala Sweden) were used for all experiments. Biotinylated capture antibodies were diluted to 0.1-0.2 μg/μL in Rexxip A Buffer and flowed over the streptavidin bead column within the microstructure of the Bioaffy CDs. The standard curve and quality control (QC) samples were prepared by spiking the hIgG4 or hFab at the range as indicated in Gyrolab™ Rexxip A Buffer mixed with 0, 4%, 10% and 25% cynomolgus monkey serum. The human IgG4 antibody hIgG4 was used to prepare the standard curve, which was a 1:4 dilution at a range between 1200-0.3 ng/ml in assay matrix.

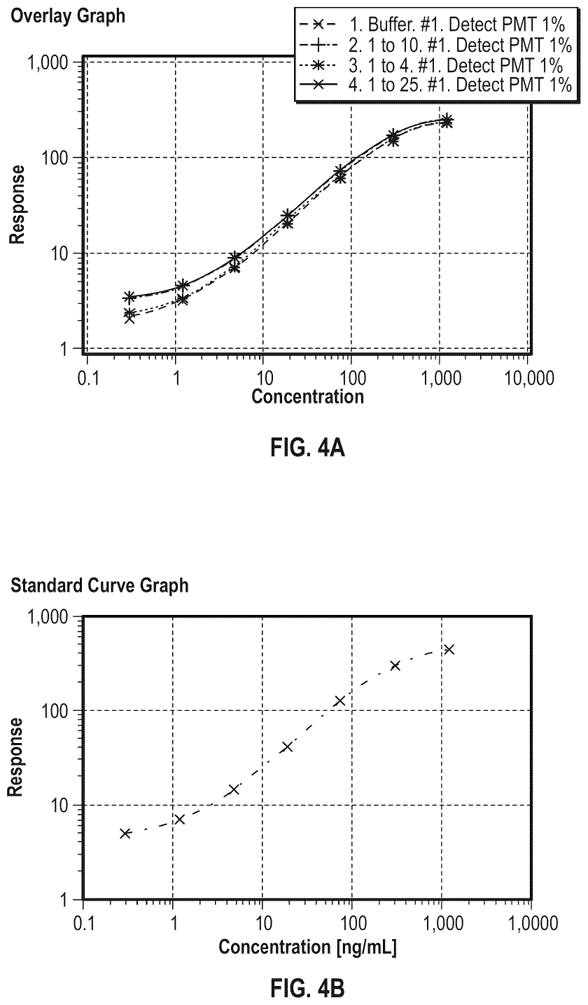

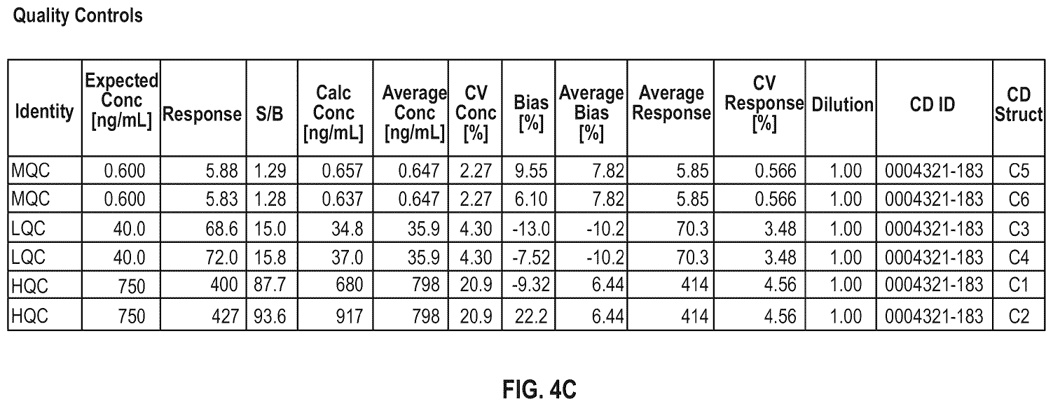

As shown in FIG. 4A, at the range of 1200-0.3 ng/ml, no matrix effect was observed at the cynomolgus monkey serum concentrations between 0 and 4%. When the assay matrix contained 10% and 25% of the cynomolgus monkey serum, some minor background signal was observed at the lower half of the standard curve. Therefore, the assay matrix containing 4% cynomolgus monkey serum was further tested with three QCs (750, 40 and 0.6 ng/ml of hIgG4) using the same standard curve. As shown in FIGS. 4B and 4C, an exemplary standard curve was obtained and two of the three QCs met both <20% bias and <20% CV, whereas the high QC had just missed the bias cut off by 0.9% (20.9%) and its CV was exemplary (4.56%).

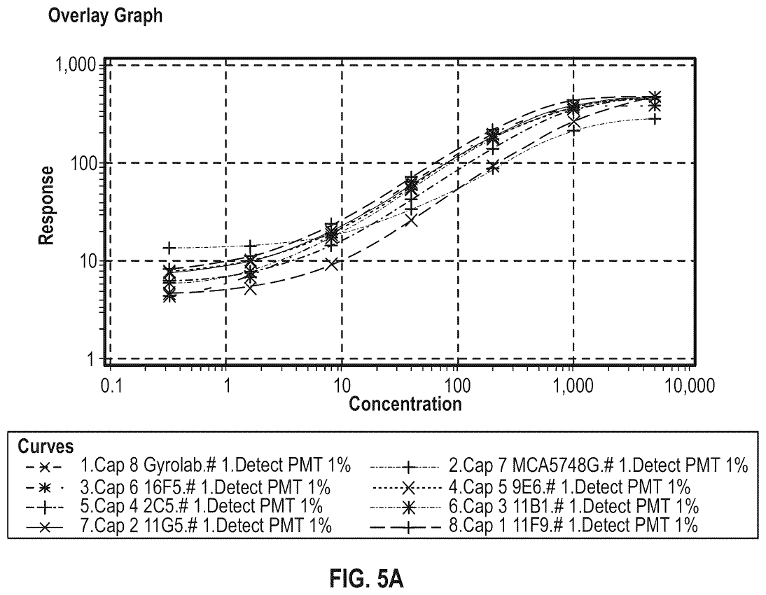

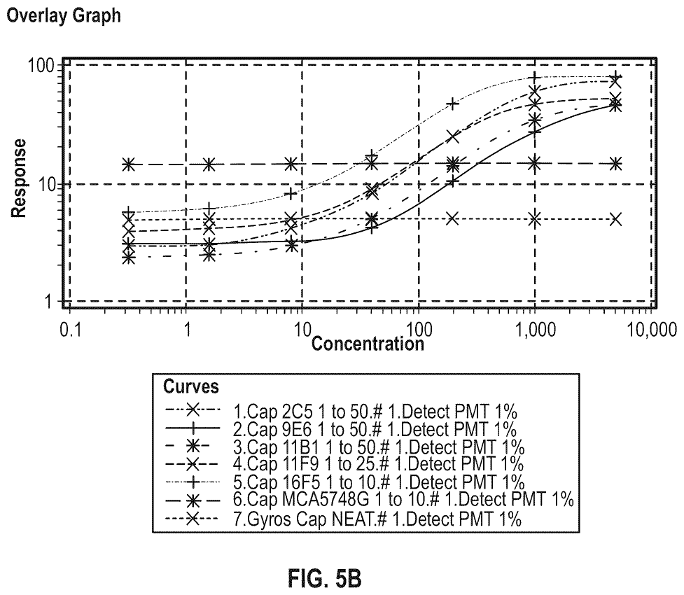

We further tested the suitability of other rabbit anti-hIgG clones to be used as the capture reagent in Gyrolab™ assay. All six rabbit anti-hIgG clones, along with the commercial mouse anti-hIgG mAb MCA5748G clone, were biotinylated and used at similar a concentration in the Gyrolab™ assay. The Gyrolab™ generic PK assay capture reagent was used as control. As shown in FIGS. 5A and 5B, when the whole antibody molecule hIgG4 was used as the standard (same range form 1200-0.3 ng/ml), all the capture reagents generated a concentration-dependent curves with the highest background seen with the commercial clone MCA5748G. However, when Fab antibody molecule hFab was used as the standard (same range form 1200-0.3 ng/ml), all six rabbit anti-hIgG capture reagents generated concentration-dependent curves as well. In contrast, both the commercial mouse anti-hIgG clone MAC5748G and the control Gyrolab™ capture reagent failed to produce concentration-dependent curves.

### Example 5: Dynamic Range of Rabbit mAb Clones in Gyrolab™ Assay

One advantage of the Gyrolab™ assay is that it offers significantly greater dynamic range compared to surface-based assay platforms such as ELISA and MSD (Fraley et al., Bioanalysis (2015) 5:1765-74). The human whole antibody hIgG4 was once again used to prepare the assay standard, which was a linear 6-point 1:5 dilution in the range of 5000-0.32 ng/ml as shown in Table 9.

TABLE 9
Performance of different capture reagents by Gyrolab ™ assay

| | Exp. | Av. | Calc | Av. | |
|---|---|---|---|---|---|
| Capture | Conc | Resp. | Conc | Bias | S/B |
| Gyrolab ™ | 0 | 4.5 | NaN | NaN | 1.0 |
| Capture | 0.32 | 4.6 | 0.4 | 20.1 | 1.0 |
| | 1.6 | 5.3 | 1.4 | −9.4 | 1.2 |
| | 8 | 9.3 | 8.3 | 3.1 | 2.1 |
| | 40 | 26.1 | 39.5 | −1.3 | 5.7 |
| | 200 | 94.1 | 201.2 | 0.6 | 20.8 |
| | 1000 | 267.3 | 997.3 | −0.3 | 59.0 |
| | 5000 | 467.6 | 5005.7 | 0.1 | 103.2 |
| MCA5748G | 0 | 11.8 | NaN | NaN | 1.0 |
| | 0.32 | 13.6 | 0.4 | 37.0 | 1.2 |
| | 1.6 | 14.2 | 1.3 | −17.1 | 1.2 |
| | 8 | 18.4 | 8.2 | 2.6 | 1.6 |
| | 40 | 33.8 | 40.4 | 1.0 | 2.9 |
| | 200 | 86.7 | 196.8 | −1.6 | 7.4 |
| | 1000 | 213.5 | 1021.5 | 2.2 | 18.1 |
| | 5000 | 282.9 | 4454.3 | −10.9 | 24.0 |
| 16F5 | 0 | 3.9 | NaN | NaN | 1.0 |
| | 0.32 | 4.4 | 0.3 | 2.2 | 1.1 |
| | 1.6 | 7.0 | 1.6 | −1.6 | 1.8 |
| | 8 | 18.5 | 8.0 | −0.1 | 4.7 |
| | 40 | 64.2 | 40.6 | 1.5 | 16.4 |
| | 200 | 197.2 | 196.0 | −2.0 | 50.5 |
| | 1000 | 368.7 | 1072.2 | 7.2 | 94.4 |
| | 5000 | 385.2 | 2171.9 | −56.6 | 98.6 |
| 9E6 | 0 | 2.9 | NaN | NaN | 1.0 |
| | 0.32 | 3.0 | 0.3 | −3.26 | 1.1 |
| | 1.6 | 4.1 | 1.6 | 1.65 | 1.4 |
| | 8 | 8.6 | 8.0 | −0.32 | 3.0 |
| | 40 | 28.4 | 39.9 | −0.16 | 9.8 |
| | 200 | 102.9 | 200.5 | 0.26 | 35.5 |
| | 1000 | 255.8 | 997.7 | −0.23 | 88.2 |
| | 5000 | 326.9 | 5041.0 | 0.82 | 112.7 |
| 2C5 | 0 | 2.1 | NaN | NaN | 1.0 |
| | 0.32 | 2.1 | 0.3 | −10.36 | 1.0 |
| | 1.6 | 3.0 | 1.7 | 6.36 | 1.4 |
| | 8 | 6.6 | 7.8 | −1.95 | 3.2 |
| | 40 | 23.4 | 40.1 | 0.18 | 11.1 |
| | 200 | 87.7 | 200.9 | 0.46 | 41.7 |
| | 1000 | 232.2 | 996.0 | −0.40 | 110.4 |
| | 5000 | 316.8 | 5043.1 | 0.86 | 150.6 |
| 19B1 | 0 | 1.7 | NaN | NaN | 1.0 |
| | 0.32 | 1.8 | 0.3 | −6.63 | 1.1 |
| | 1.6 | 2.7 | 1.7 | 4.82 | 1.6 |
| | 8 | 6.2 | 7.8 | −2.32 | 3.6 |
| | 40 | 22.8 | 40.5 | 1.20 | 13.3 |
| | 200 | 82.9 | 199.2 | −0.39 | 48.3 |
| | 1000 | 202.9 | 1001.9 | 0.19 | 118.2 |
| | 5000 | 246.3 | 4936.0 | −1.28 | 143.6 |
| 11G5 | 0 | 7.4 | NaN | NaN | 1.0 |
| | 0.32 | 7.5 | 0.4 | 10.6 | 1.0 |
| | 1.6 | 9.8 | 1.5 | −5.3 | 1.3 |
| | 8 | 20.3 | 8.0 | 0.6 | 2.7 |
| | 40 | 60.9 | 40.6 | 1.6 | 8.2 |
| | 200 | 183.7 | 196.0 | −2.0 | 24.8 |
| | 1000 | 393.7 | 1025.2 | 2.5 | 53.1 |
| | 5000 | 468.5 | 4581.7 | −8.4 | 63.3 |
| 11F9 | 0 | 3.0 | NaN | NaN | 1.0 |
| | 0.32 | 3.1 | 0.3 | −4.19 | 1.0 |
| | 1.6 | 5.0 | 1.7 | 3.47 | 1.7 |
| | 8 | 12.8 | 7.9 | −1.27 | 4.3 |
| | 40 | 45.1 | 39.8 | −0.47 | 15.2 |
| | 200 | 148.5 | 202.9 | 1.45 | 50.2 |
| | 1000 | 289.3 | 972.7 | −2.73 | 97.8 |
| | 5000 | 329.5 | 8125.0 | 62.50 | 111.4 |

Exp.: expected.
Av.: average.
Resp.: response.
Conc: concentration.

A similar Gyrolab™ assay procedure was performed and the data was then extracted and compared. At the upper end (5000 ng/ml), the Gyrolab™ capture and the rabbit mAb clones 9E6, 2C5 and 19B1 all had acceptable biases (<20) and signal-to-noise ratios greater than 100. The three rabbit anti-hIgG clones showed better signal-to-noise ratios than the Gyrolab™ capture. At 5000 ng/ml high end point, although clone 11G5 had acceptable average bias, its signal-to-noise ratio was low (63.3%), which was caused by the relatively high blank signal level (7.4). For the same reason, the commercial mAb clone MCA5748 also had a very poor signal-to-noise ratio at the upper end (24.0).

These data suggest that clones 9E6, 2C5 and 19B1 offer a similar dynamic range when compared to the Gyrolab™ capture reagent with better signal to noise ratios. The detection dynamic range by clones 9E6, 2C5 and 19B1 was significantly better than the commercial mouse anti-hIgG clone MCA5748G. Clones 16F5, and 11F9 performed well at the 0-1000 ng/ml range, with <20% average Bias, but not at the 5000 ng/ml data point. This might be due to the fact that these two clones possess different binding epitopes from the four other clones.

Thus, in the initial Gyrolab™ assay, when the experiment was performed using clone 16F5 as the capture reagent, concentration-dependent assay curves were generated with the assay matrix containing up to 25% cynomolgus monkey serum. The assay performed well in the range of 0.30-1200 ng/ml with the assay matrix containing 4% cynomolgus monkey serum. In the expanded Gyrolab™ evaluation assays, all the six rabbit mAb clones demonstrated their capability to serve as capture reagents for the detection of both whole human IgG and Fab molecules. However, the two control capture reagents, the Gyros capture reagent and the commercial clone MCA5748G, were only able to detect the whole human IgG, but not the Fab molecule.

As summarized in Table 10, we developed six rabbit anti-human IgG mAb clones that can bind to both the whole human IgG molecule and the Fab molecule, without binding to cynomolgus monkey IgG. These six clones belong to three different epitope groups and are different from the commercial clone MCA5748G. Each of these clones have been tested for detection of human IgGs and Fabs in Gyrolab™ assays, and three of them exhibited greater dynamic range and signal to noise ratio.

TABLE 10
Summary of six rabbit anti-hIgG clones

| Antibody | | | | | VH |
|---|---|---|---|---|---|
| clones | hIgG | hFab | cynoIgG | Epitope | Phylogenetic Tree |
| Gyros Capture | Yes | No | No | Unknow | N/A |
| MCA5748G | Yes | No | minor | Unknow | N/A |
| 16F5 | Yes | Yes | No | Group A | Group A |
| 11F9 | Yes | Yes | No | Group B | Group B |
| 9E6 | Yes | Yes | No | Group C | Group C |
| 2C5 | Yes | Yes | No | Group C | Group C |
| 19B1 | Yes | Yes | No | Group C | Group C |
| 11G5 | Yes | Yes | No | Group C | Group C |

### Example 6: Use of Rabbit Anti-Human IgG Monoconal Antibody Clones as Generic Positive Control for PandA Assay

Rabbit polyclonal anti-drug antibodies (pAb) are commonly used as the performance controls for anti-drug antibody (ADA) detection. But the generation of pAb is time consuming and requires repetitive use of experimental animals. Furthermore, pAb often shows low sensitivity as a control. This Example describes a study comparing two recombinantly produced rabbit anti-human IgG mAbs, 2C5 and 11G5, and a commercial mouse anti-Fc mAb (JDC-10; Southern Biotech Cat #9040-01) as a positive control in a PandA assay.

2C5 and 11G5, along with JDC-10, were diluted in monkey plasma pool at 5 μg/ml, 1 μg/ml, 0.75 μg/ml, 0.5 μg/ml, and 0 μg/ml, respectively.

Plasma samples containing diluted antibody were initially diluted 1:5 in assay buffer (300 mM Acetic acid, 2% BSA) containing excess drug (a monoclonal antibody of human IgG4 isotype; 10-50 μg/mL) and incubated for one hour at 37° C. with 450 rpm in a polypropylene plate to allow complexes to form between the drug and the added antibody in the sample. This was followed by the addition of 3% PEG in borate (pH 8.0) to each sample and an overnight incubation at 2-8° C. The final concentration of PEG buffer in each sample was 1.5%.

On the following day, the plate was centrifuged at 4,000 rpm for 20 minutes to precipitate the complexes into a pellet. The pellet was re-suspended with 1.5% PEG in borate (pH 8.0) and centrifuged for a second time at 4,000 rpm for 20 minutes. The wash cycle was repeated three times. Following the final centrifugation, each sample was suspended in 100 μl of 300 mM acetic acid and further diluted 1:10 (20 μl sample+180 μl acetic acid) for a final sample dilution of 1:50. Diluted samples were added in duplicate to the wells of an MSD High Bind plate at 25 μl per well and incubated for one hour at 24° C. with shaking at 450 rpm.

Following the incubation, the plate was washed with 1× plate wash buffer and blocked with 3% milk in PBS for one hour at 24° C. with shaking. The plate was then washed and 100 ng/ml of sulfo-TAG-Drug was added to the samples and incubated for one hour at 24° C. with shaking. After the final incubation, the plate was washed with 0.05% Tween in PBS. Read buffer T 2× was then added and the plate was read on a Sector PR2400. The electrochemiluminescence (ECL) signal was proportional to the anti-drug antibody in each sample.

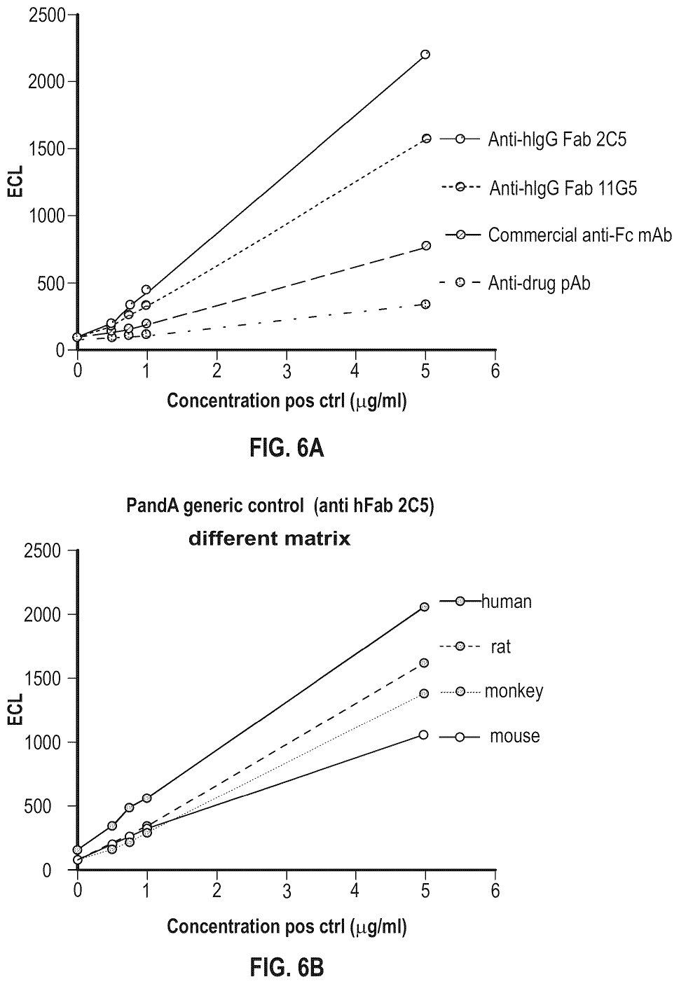

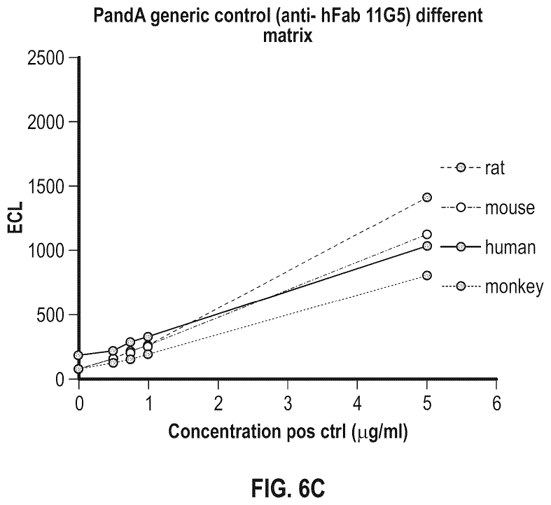

As shown in FIG. 6A and Table 11, the two rabbit anti-hIgG clones 2C5 and 11G5 demonstrated a greater dynamic range and sensitivity compared to the commercial anti-Fc mAb clone JDC-10.

TABLE 11
Comparison of Different Antibodies as Positive Control in PandA

| | ECL Signal | | |
|---|---|---|---|
| | Anti-Fc | Anti-hFab | Anti-hFab |
| PC (μg/ml) | mAb JDC-10 | mAb 2C5 | mAb 11G5 |
| 5 | 755.5 | 2,180 | 1,551 |
| 1 | 182.5 | 426 | 319.5 |
| 0.75 | 148.5 | 323 | 257 |
| 0.5 | 130.5 | 187.5 | 171 |
| NC | 95 | 94.5 | 95 |
| S/N | 1.37 | 1.98 | 1.80 |

*PC: positive control.
S/N: signal to noise.
NC: negative control.

Clones 2C5 and 11G5 also served as performance controls and were tested in human, rat, monkey, and mouse plasma and serum matrices. The data show that both 2C5 (FIG. 6B) and 11G5 (FIG. 6C) could distinguish between human, monkey, mouse, and rat sera, with 2C5 being more sensitive than 11G5 (Table 12). In the human matrix, the background increased for both clones as expected.

TABLE 12
Sensitivity in PandA Assay

| | S/N (monkey) | |
|---|---|---|
| PC (μg/ml) | 2C5 | 11G5 |
| 5 | 15.70 | 9.76 |
| 1 | 3.39 | 2.40 |
| 0.75 | 2.51 | 1.92 |
| 0.5 | 1.93 | 1.58 |

The above results demonstrate that recombinantly produced rabbit anti-human IgG mAbs are an excellent generic positive control for preclinical assays, especially for the PandA format (e.g., for monkey, rat, and mouse matrices). These antibodies performed well in both plasma and serum matrices. The rabbit IgG mAbs have an improved dynamic range and assay sensitivity.

1. (canceled)

2. An isolated monoclonal antibody or an antigen-binding portion thereof that binds specifically to human IgG, wherein the antibody or portion comprises heavy chain variable domain (VH) and light chain variable domain (VL) respectively comprising
SEQ ID NOs: 13 and 19,
SEQ ID NOs: 14 and 20,
SEQ ID NOs: 15 and 21,
SEQ ID NOs: 16 and 22,
SEQ ID NOs: 17 and 23, or
SEQ ID NOs: 18 and 24.

3. The monoclonal antibody of claim 2, wherein the antibody is a rabbit IgG antibody.

4. The monoclonal antibody of claim 2, comprising a heavy chain constant region amino acid sequence of SEQ ID NO: 25, and/or a light chain constant region amino sequence of SEQ ID NO: 26.

5-20. (canceled)

21. The monoclonal antibody of claim 2, wherein the heavy chain variable domain (VH) and light chain variable domain (VL) respectively comprise SEQ ID NOs: 13 and 19.

22. The monoclonal antibody of claim 2, wherein the heavy chain variable domain (VH) and light chain variable domain (VL) respectively comprise SEQ ID NOs: 14 and 20.

23. The monoclonal antibody of claim 2, wherein the VH and VL respectively comprise SEQ ID NOs: 15 and 21.

24. The monoclonal antibody of claim 2, wherein the VH and VL respectively comprise SEQ ID NOs: 16 and 22.

25. The monoclonal antibody of claim 2, wherein the VH and VL respectively comprise SEQ ID NOs: 17 and 23.

26. The monoclonal antibody of claim 2, wherein the VH and VL respectively comprise SEQ ID NOs: 18 and 24.

27. The monoclonal antibody of claim 2, comprising a heavy chain and a light chain having the amino acid sequences of
SEQ ID NOs: 63 and 69,
SEQ ID NOs: 64 and 70,
SEQ ID NOs: 65 and 71,
SEQ ID NOs: 66 and 72,
SEQ ID NOs: 67 and 73, or
SEQ ID NOs: 68 and 74,
respectively, with or without the leader sequences.

28. The monoclonal antibody or antigen-binding portion of claim 2, further comprising a detectable label.

29. A composition or a kit comprising the monoclonal antibody or antigen-binding portion of claim 2 in an aqueous buffered solution.

30. An isolated nucleic acid molecule encoding the heavy chain, the light chain, or both, of the monoclonal antibody or antigen-binding portion of claim 2.

31. The nucleic acid molecule of claim 30, comprising SEQ ID NO: 1, 2, 3, 4, 5, 6, 7, 8, 9, 10, 11, or 12.

32. The nucleic acid molecule of claim 31, comprising
SEQ ID NOs: 1 and 7,
SEQ ID NOs: 2 and 8,
SEQ ID NOs: 3 and 9,
SEQ ID NOs: 4 and 10,
SEQ ID NOs: 5 and 11, or
SEQ ID NOs: 6 and 12.

33. An expression construct comprising the nucleic acid molecule of claim 30.

34. A host cell comprising nucleotide sequences encoding the heavy chain and the light chain of the monoclonal antibody or antigen-binding portion of claim 2.

35. The host cell of claim 34, wherein the host cell is a mammalian cell.

36. A method of producing an antibody or an antigen-binding portion thereof, comprising:
culturing the host cell of claim 34 under conditions that allow expression of the heavy chain and light chain of the antibody or portion, and
isolating the antibody or portion from the cell culture.

37. A method of detecting human IgG or a fragment thereof in a sample, comprising contacting the sample with an antibody or antigen-binding portion of claim 2.

38. The method of claim 37, wherein the sample is obtained from an animal that has been administered an antibody comprising a human IgG constant region or a fragment thereof.

39. The method of claim 37, wherein the human IgG constant region is a human IgG1, IgG2, IgG3, or IgG4 constant region.

40. The method of claim 37, wherein the animal has been administered a Fab or F(ab′)2 fragment of an antibody comprising a human IgG1, IgG2, IgG3, or IgG4 constant region.

41. The method of claim 37, wherein the sample is a tissue sample.

42. The method of claim 41, wherein the sample is a blood, serum, or plasma sample.

43. The method of claim 37, wherein the animal is a non-human primate.

44. The method of claim 43, wherein the non-human primate is a cynomolgus monkey or rhesus monkey.


SEQUENCE LISTING (74 sequences)

SEQ ID NO: 1 - DNA, 1392 bp, synthetic construct
  misc_feature 1..1392: note = Description of Artificial Sequence: Synthetic polynucleotide
  source 1..1392: mol_type = other DNA
atggagactgggctgcgctggcttctcctggtcactgtgctcaaaggtgtccagtgtcaggagcagctggtggagtccgggggagacctggtcaagcctggggcatccctgacactcacctgcacagcctctggattctccttcagtagcggctactacatgtgctgggtccgccaggctccagggaaggggctggagtggatcgcatgcatttatggtggtgcgcttactaatacttactacgcgacctgggcgaaaggccgattcaccatctccaagacctcgtcgaccacggtgaccctgcaaatgaccagtctgacagccgcggacacggccacctatttctgtgcgagagatctgggtgctgctggtgatgcttataacttgtgggggccaggcaccctggtcaccgtctcctcagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccatgtgcccaccccctgaactcccggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccgaccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatag

SEQ ID NO: 2 - DNA, 1386 bp, synthetic construct
  misc_feature 1..1386: note = Description of Artificial Sequence: Synthetic polynucleotide
  source 1..1386: mol_type = other DNA
atggagactgggctgcgctggcttctcctggtcgctgtgctcaaaggtgtccagtgtcagtcgttggaggagtccgggggagacctggtcaagcctggggcatccctgacactcacctgcaaagcctctggattcgacttcagtagcagctactacatgtgctgggtccgccaggctccagggagactggagtggatcgcatgcatttatggtggtggtctgagtaacacttactacgcgggctgggcaaaaggccgattcaccatctccaaaacctcgtcgaccacggtgactctgcaaatgaccagtctgacagtcgcggacacggccacctatttctgtgcgagagatgctgggactagtggtgattaccttaacttgtggggcccgggcaccctggtcaccgtctcctcagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccatgtgcccaccccctgaactcccggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccgaccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatag

SEQ ID NO: 3 - DNA, 1389 bp, synthetic construct
  misc_feature 1..1389: note = Description of Artificial Sequence: Synthetic polynucleotide
  source 1..1389: mol_type = other DNA
atggagactgggctgcgctggcttctcctggtcgctgtgctcaaaggtgtccagtgtcagcagcagctggaggagtccgggggaggcctggtccagcctgagggatccctgacactcacctgcatagcttctggattctccttcagtagcagccactggatatgttgggtccgccaggctccagggaaggggctggagtggatcgcatgcatgtctactagtagtggtagcacttacgatgcgaactgggcgaaaggccgattcaccatctccaaaacctcgtcgaccacggtgactctgcaaatgaccagtctgacagccgcggacacggccacctatttctgtgcgagagatgttggcggtagtactacttactttgacttgtggggcccaggcaccctggtcaccgtctcctcagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccatgtgcccaccccctgaactcccggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccgaccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatag

SEQ ID NO: 4 - DNA, 1389 bp, synthetic construct
  misc_feature 1..1389: note = Description of Artificial Sequence: Synthetic polynucleotide
  source 1..1389: mol_type = other DNA
atggagactgggctgcgctggcttctcctggtcgctgtgctcaaaggtgtccagtgtcagtcgttggaggagtccgggggagacctggtcaagcctggggcatccctgacactcacctgcacagcttctggattctccttcagtagcagctactacatgtgctgggtccgccaggctccagggaagaggctggagtggatcgcttgcatttatggtggtggtctgagtaacacttactacgcgggctgggcaaaaggccgattcaccatctccaaaacctcgtcgaccacggtgactctgcaaatgaccagtctgacagccgcggacacggccacctatttctgtgcgagagatgctgggactagtggtgattaccttaacttgtggggcccaggcaccctggtcaccgtctcctcagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccatgtgcccaccccctgaactcccggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccgaccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatag

SEQ ID NO: 5 - DNA, 1398 bp, synthetic construct
  misc_feature 1..1398: note = Description of Artificial Sequence: Synthetic polynucleotide
  source 1..1398: mol_type = other DNA
atggagactgggctgcgctggcttctcctggtcgctgtgctcaaaggtgtccagtgtcagcagcagttggaggagtccgggggaggcctggtcaagcctggaggaaccctgacactcacctgcaaagcctctggaatcgacttcagtaactactactacatgtgctgggtccgccaggctccagggaaggggctggagttgatcgcatgcatttatactggtagtagtggtagcacatggtacgcgacctgggcgaagggccgattcaccatctccaaaacctcgtcgaccacggtgactctgcaaatgaccagtctgacagccgcggacacggccacctatttctgtgcgagagatcgtgatgttggtagtctttatgactccttagatctctggggccagggcaccctggtcaccgtctctccagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccatgtgcccaccccctgaactcccggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccgaccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatag

SEQ ID NO: 6 - DNA, 1392 bp, synthetic construct
  misc_feature 1..1392: note = Description of Artificial Sequence: Synthetic polynucleotide
  source 1..1392: mol_type = other DNA
atggagactgggctgcggtggcttctcctggtcgctgtgctcaaaggtgtccagtgtcaggagcagctggtggagtccgggggaggcctggtccagcctgagggatccctgacactcacctgcacagcttctggattctccttcagtgacagctactacatgtgctgggtccgccaggctccagggaaggggctggagtggatcgcatgcatttatggtggtactattactaatacttactacgcgagctgggcgaaaggccgattcaccatctccaagacctcgtcgaccacggtgaccctgcaaatgaccagtctgacagccgcggacacggccacctatttctgtgcgagagatctgggtgctgctggtgatgcttataacttgtggggcccaggcaccctggtcaccgtctcctcagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccatgtgcccaccccctgaactcccggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccgaccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatag

SEQ ID NO: 7 - DNA, 711 bp, synthetic construct
  misc_feature 1..711: note = Description of Artificial Sequence: Synthetic polynucleotide
  source 1..711: mol_type = other DNA
atggacacgagggcccccactcagctgctggggctcctgctgctctggctcccaggtgccagatgtgcctctgatatgacccagactccagcctccgtgtctgcagctgtgggaggcacagtcaccatcaagtgccaggccagtgagagcatttacagtggtttggcctggtatcagcagaaaccagggcagcctcccaagctcctgatctttgatgcatccgatctggcatctggggtcccatcgcggttcaaaggcagtagatctgagacagagtacactctcaccatcagcgacctggagtgtgccgatgctgccacttactactgtcaatgcactgatcgtaatagtattacttcttatgctttcggcggagggaccgaggtggtggtcaaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 8 - DNA, 717 bp, synthetic construct
  misc_feature 1..717: note = Description of Artificial Sequence: Synthetic polynucleotide
  source 1..717: mol_type = other DNA
atggacacgagggcccccactcagctgctggggctcctgctgctctggctcccaggtgccagatgtgccgtcgtgatgacccagactgcatcccccgtgtctggagctgtgggaggcacagtcaccatcaattgccaggccagtcagagtattagtagtagctacttatcctggtatcagcagaaaccagggcagcctcccaagctcctgatctatggtgcatccactctggcatctggggtcccatcgcggttcaaaggcagtggatctgggacacagttcactctcaccatcagtggcgtgcagtgtgacgatgctgccacttactactgtgcatacgatgcttatcgtctcagtagtcctgataatattttcggcggagggaccgaggtggtggtcaaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 9 - DNA, 711 bp, synthetic construct
  misc_feature 1..711: note = Description of Artificial Sequence: Synthetic polynucleotide
  source 1..711: mol_type = other DNA
atggacacgagggcccccactcagctgctggggctcctgctgctctggctcccaggtgccagatgtgcctatgatatgacccagactccagcctccgtgtctgaacctgtgggaggcacagtcaccatcaagtgccaggccagtcagagcattagcaatgaattatcctggtatcaacaaaaaccagggcagcctcccaaactcctgatctacagggcatccactctggcatctggggtcccatcgcggttcaaaggcagtggatctgggacacagttcactctcaccatcaacggcgtggagtgtgccgatgctgccacttactactgtcaacagggttatagtattagtaatgttgataatactttcggcggagggaccgaggtggtggtcaaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 10 - DNA, 717 bp, synthetic construct
  misc_feature 1..717: note = Description of Artificial Sequence: Synthetic polynucleotide
  source 1..717: mol_type = other DNA
atggacacgagggcccccactcagctgctggggctcctgctgctctggctcccaggtgccagatgtgccgtcgtgatgacccagactgcatcccccgtgtctggagctgtgggaggcacagtcaccatcaattgccaggccagtcagagtattagtgctagcgccttatcctggtatcaacagaaaccagggcagcctcccaagctcctgatctatgctgcatccactctggaatctggggtcccatcgcggttcaaaggcagtggatctgggacacagttcactctcaccatcagcggcgtgcagtgtgacgatgctgccacttactactgtgcatacgatggttatcgtctcagtagtgctgataatattttcggcggagggaccgaggtggtggtcaaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 11 - DNA, 711 bp, synthetic construct
  misc_feature 1..711: note = Description of Artificial Sequence: Synthetic polynucleotide
  source 1..711: mol_type = other DNA
atggacacgagggcccccactcagctgctggggctcctgctgctctggctcccaggtgccagatgtgcctatgatatgacccagactccagcctccgtggaggcagctgtgggaggcacagtcaccatcaattgccaggccagtcagagcattaataattggttatcctggtatcagcagaaaccagggcagcgtcccaagctcctgatttaccaggcatccactctggcatctggggtctcatcgcggttcaaaggcagtggatctgggacacactttactctcaccatcagcgacctggagtgtgccgatgctgccacttactactgtcaacagggttggagtatagacgatattgataatgctttcggcggagggaccgaggtggtggtcaagggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 12 - DNA, 711 bp, synthetic construct
  misc_feature 1..711: note = Description of Artificial Sequence: Synthetic polynucleotide
  source 1..711: mol_type = other DNA
atggacacgagggcccccactcagctgctggggctcctgctgctctggctcccaggtgccagatgtgcctctgatatgacccagactccagcctccgtgtctgaacctgtgggaggcacagtcaccatcaagtgccaggccagtgagaacatttacagctctttagcctggtatcagcagaaaccagggcagcctcccaagctcctgatctatgatgcatccaatctggcatctggggtcccatcgcggttcagcggcagtggatctgggacagagttcactctcaccatcagcgacctggagtgtgccgatgctgccacttactactgtcaatgcacttatcgtagtagtagtagttcttatgctttcggcggagggaccgaggtggtggtcaaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 13 - protein, 121 aa, synthetic construct
  REGION 1..121: note = Description of Artificial Sequence: Synthetic polypeptide
QEQLVESGGDLVKPGASLTLTCTASGFSFSSGYYMCWVRQAPGKGLEWIACIYGGALTNTYYATWAKGRFTISKTSSTTVTLQMTSLTAADTATYFCARDLGAAGDAYNLWGPGTLVTVSS

SEQ ID NO: 14 - protein, 119 aa, synthetic construct
  REGION 1..119: note = Description of Artificial Sequence: Synthetic polypeptide
QSLEESGGDLVKPGASLTLTCKASGFDFSSSYYMCWVRQAPGRLEWIACIYGGGLSNTYYAGWAKGRFTISKTSSTTVTLQMTSLTVADTATYFCARDAGTSGDYLNLWGPGTLVTVSS

SEQ ID NO: 15 - protein, 120 aa, synthetic construct
  REGION 1..120: note = Description of Artificial Sequence: Synthetic polypeptide
QQQLEESGGGLVQPEGSLTLTCIASGFSFSSSHWICWVRQAPGKGLEWIACMSTSSGSTYDANWAKGRFTISKTSSTTVTLQMTSLTAADTATYFCARDVGGSTTYFDLWGPGTLVTVSS

SEQ ID NO: 16 - protein, 119 aa, synthetic construct
  REGION 1..119: note = Description of Artificial Sequence: Synthetic polypeptide
QSLEESGGDLVKPGASLTLTCTASGFSFSSSYYMCWVRQAPGKRLEWIACIYGGGLSNTYYAGWAKGRFTISKTSSTTVTLQMTSLTAADTATYFCARDATSGDYLNLWGPGTLVTVSS

SEQ ID NO: 17 - protein, 123 aa, synthetic construct
  REGION 1..123: note = Description of Artificial Sequence: Synthetic polypeptide
QQQLEESGGGLVKPGGTLTLTCKASGIDFSNYYYMCWVRQAPGKGLELIACIYTGSSGSTWYATWAKGRFTISKTSSTTVTLQMTSLTAADTATYFCARDRDVGSLYDSLDLWGQGTLVTVSP

SEQ ID NO: 18 - protein, 121 aa, synthetic construct
  REGION 1..121: note = Description of Artificial Sequence: Synthetic polypeptide
QEQLVESGGGLVQPEGSLTLTCTASGFSFSDSYYMCWVRQAPGKGLEWIACIYGGTITNTYYASWAKGRFTISKTSSTTVTLQMTSLTAADTATYFCARDLGAAGDAYNLWGPGTLVTVSS

SEQ ID NO: 19 - protein, 110 aa, synthetic construct
  REGION 1..110: note = Description of Artificial Sequence: Synthetic polypeptide
ASDMTQTPASVSAAVGGTVTIKCQASESIYSGLAWYQQKPGQPPKLLIFDASDLASGVPSRFKGSRSETEYTLTISDLECADAATYYCQCTDRNSITSYAFGGGTEVVVK

SEQ ID NO: 20 - protein, 112 aa, synthetic construct
  REGION 1..112: note = Description of Artificial Sequence: Synthetic polypeptide
AVVMTQTASPVSGAVGGTVTINCQASQSISSSYLSWYQQKPGQPPKLLIYGASTLASGVPSRFKGSGSGTQFTLTISGVQCDDAATYYCAYDAYRLSSPDNIFGGGTEVVVK

SEQ ID NO: 21 - protein, 110 aa, synthetic construct
  REGION 1..110: note = Description of Artificial Sequence: Synthetic polypeptide
AYDMTQTPASVSEPVGGTVTIKCQASQSISNELSWYQQKPGQPPKLLIYRASTLASGVPSRFKGSGSGTQFTLTINGVECADAATYYCQQGYSISNVDNTFGGGTEVVVK

SEQ ID NO: 22 - protein, 112 aa, synthetic construct
  REGION 1..112: note = Description of Artificial Sequence: Synthetic polypeptide
AVVMTQTASPVSGAVGGTVTINCQASQSISASALSWYQQKPGQPPKLLIYAASTLESGVPSRFKGSGSGTQFTLTISGVQCDDAATYYCAYDGYRLSSADNIFGGGTEVVVK

SEQ ID NO: 23 - protein, 110 aa, synthetic construct
  REGION 1..110: note = Description of Artificial Sequence: Synthetic polypeptide
AYDMTQTPASVEAAVGGTVTINCQASQSINNWLSWYQQKPGQRPKLLIYQASTLASGVSSRFKGSGSGTHFTLTISDLECADAATYYCQQGWSIDDIDNAFGGGTEVVVK

SEQ ID NO: 24 - protein, 110 aa, synthetic construct
  REGION 1..110: note = Description of Artificial Sequence: Synthetic polypeptide
ASDMTQTPASVSEPVGGTVTIKCQASENIYSSLAWYQQKPGQPPKLLIYDASNLASGVPSRFSGSGSGTEFTLTISDLECADAATYYCQCTYRSSSSSYAFGGGTEVVVK

SEQ ID NO: 25 - protein, 323 aa, synthetic construct
  REGION 1..323: note = Description of Artificial Sequence: Synthetic polypeptide
GQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELPGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPTVLDSDGSYFLYSKLSVPTSEWQRGDVFTCSVMHEALHNHYTQKSISRSPGK

SEQ ID NO: 26 - protein, 104 aa, synthetic construct
  REGION 1..104: note = Description of Artificial Sequence: Synthetic polypeptide
GDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 27 - protein, 10 aa, synthetic construct
  REGION 1..10: note = Description of Artificial Sequence: Synthetic peptide
FSFSSGYYMC

SEQ ID NO: 28 - protein, 19 aa, synthetic construct
  REGION 1..19: note = Description of Artificial Sequence: Synthetic peptide
WIACIYGGALTNTYYATWA

SEQ ID NO: 29 - protein, 12 aa, synthetic construct
  REGION 1..12: note = Description of Artificial Sequence: Synthetic peptide
ARDLGAAGDAYN

SEQ ID NO: 30 - protein, 8 aa, synthetic construct
  REGION 1..8: note = Description of Artificial Sequence: Synthetic peptide
ESIYSGLA

SEQ ID NO: 31 - protein, 11 aa, synthetic construct
  REGION 1..11: note = Description of Artificial Sequence: Synthetic peptide
LLIFDASDLAS

SEQ ID NO: 32 - protein, 12 aa, synthetic construct
  REGION 1..12: note = Description of Artificial Sequence: Synthetic peptide
QCTDRNSITSYA

SEQ ID NO: 33 - protein, 10 aa, synthetic construct
  REGION 1..10: note = Description of Artificial Sequence: Synthetic peptide
FDFSSSYYMC

SEQ ID NO: 34 - protein, 19 aa, synthetic construct
  REGION 1..19: note = Description of Artificial Sequence: Synthetic peptide
WIACIYGGGLSNTYYAGWA

SEQ ID NO: 35 - protein, 12 aa, synthetic construct
  REGION 1..12: note = Description of Artificial Sequence: Synthetic peptide
ARDAGTSGDYLN

SEQ ID NO: 36 - protein, 9 aa, synthetic construct
  REGION 1..9: note = Description of Artificial Sequence: Synthetic peptide
QSISSSYLS

SEQ ID NO: 37 - protein, 11 aa, synthetic construct
  REGION 1..11: note = Description of Artificial Sequence: Synthetic peptide
LLIYGASTLAS

SEQ ID NO: 38 - protein, 13 aa, synthetic construct
  REGION 1..13: note = Description of Artificial Sequence: Synthetic peptide
AYDAYRLSSPDNI

SEQ ID NO: 39 - protein, 10 aa, synthetic construct
  REGION 1..10: note = Description of Artificial Sequence: Synthetic peptide
FSFSSSHWIC

SEQ ID NO: 40 - protein, 18 aa, synthetic construct
  REGION 1..18: note = Description of Artificial Sequence: Synthetic peptide
WIACMSTSSGSTYDANWA

SEQ ID NO: 41 - protein, 12 aa, synthetic construct
  REGION 1..12: note = Description of Artificial Sequence: Synthetic peptide
ARDVGGSTTYFD

SEQ ID NO: 42 - protein, 8 aa, synthetic construct
  REGION 1..8: note = Description of Artificial Sequence: Synthetic peptide
QSISNELS

SEQ ID NO: 43 - protein, 11 aa, synthetic construct
  REGION 1..11: note = Description of Artificial Sequence: Synthetic peptide
LLIYRASTLAS

SEQ ID NO: 44 - protein, 12 aa, synthetic construct
  REGION 1..12: note = Description of Artificial Sequence: Synthetic peptide
QQGYSISNVDNT

SEQ ID NO: 45 - protein, 10 aa, synthetic construct
  REGION 1..10: note = Description of Artificial Sequence: Synthetic peptide
FSFSSSYYMC

SEQ ID NO: 46 - protein, 19 aa, synthetic construct
  REGION 1..19: note = Description of Artificial Sequence: Synthetic peptide
WIACIYGGGLSNTYYAGWA

SEQ ID NO: 47 - protein, 12 aa, synthetic construct
  REGION 1..12: note = Description of Artificial Sequence: Synthetic peptide
ARDAGTSGDYLN

SEQ ID NO: 48 - protein, 9 aa, synthetic construct
  REGION 1..9: note = Description of Artificial Sequence: Synthetic peptide
QSISASALS

SEQ ID NO: 49 - protein, 11 aa, synthetic construct
  REGION 1..11: note = Description of Artificial Sequence: Synthetic peptide
LLIYAASTLES

SEQ ID NO: 50 - protein, 13 aa, synthetic construct
  REGION 1..13: note = Description of Artificial Sequence: Synthetic peptide
AYDGYRLSSADNI

SEQ ID NO: 51 - protein, 10 aa, synthetic construct
  REGION 1..10: note = Description of Artificial Sequence: Synthetic peptide
IDFSNYYYMC

SEQ ID NO: 52 - protein, 19 aa, synthetic construct
  REGION 1..19: note = Description of Artificial Sequence: Synthetic peptide
LIACIYTGSSGSTWYATWA

SEQ ID NO: 53 - protein, 14 aa, synthetic construct
  REGION 1..14: note = Description of Artificial Sequence: Synthetic peptide
ARDRDVGSLYDSLD

SEQ ID NO: 54 - protein, 8 aa, synthetic construct
  REGION 1..8: note = Description of Artificial Sequence: Synthetic peptide
QSINNWLS

SEQ ID NO: 55 - protein, 11 aa, synthetic construct
  REGION 1..11: note = Description of Artificial Sequence: Synthetic peptide
LLIYQASTLAS

SEQ ID NO: 56 - protein, 12 aa, synthetic construct
  REGION 1..12: note = Description of Artificial Sequence: Synthetic peptide
QQGWSIDDIDNA

SEQ ID NO: 57 - protein, 10 aa, synthetic construct
  REGION 1..10: note = Description of Artificial Sequence: Synthetic peptide
FSFSDSYYMC

SEQ ID NO: 58 - protein, 19 aa, synthetic construct
  REGION 1..19: note = Description of Artificial Sequence: Synthetic peptide
WIACIYGGTITNTYYASWA

SEQ ID NO: 59 - protein, 12 aa, synthetic construct
  REGION 1..12: note = Description of Artificial Sequence: Synthetic peptide
ARDLGAAGDAYN

SEQ ID NO: 60 - protein, 8 aa, synthetic construct
  REGION 1..8: note = Description of Artificial Sequence: Synthetic peptide
ENIYSSLA

SEQ ID NO: 61 - protein, 11 aa, synthetic construct
  REGION 1..11: note = Description of Artificial Sequence: Synthetic peptide
LLIYDASNLAS

SEQ ID NO: 62 - protein, 12 aa, synthetic construct
  REGION 1..12: note = Description of Artificial Sequence: Synthetic peptide
QCTYRSSSSSYA

SEQ ID NO: 63 - protein, 463 aa, synthetic construct
  REGION 1..463: note = Description of Artificial Sequence: Synthetic polypeptide
METGLRWLLLVTVLKGVQCQEQLVESGGDLVKPGASLTLTCTASGFSFSSGYYMCWVRQAPGKGLEWIACIYGGALTNTYYATWAKGRFTISKTSSTTVTLQMTSLTAADTATYFCARDLGAAGDAYNLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELPGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPTVLDSDGSYFLYSKLSVPTSEWQRGDVFTCSVMHEALHNHYTQKSISRSPGK

SEQ ID NO: 64 - protein, 461 aa, synthetic construct
  REGION 1..461: note = Description of Artificial Sequence: Synthetic polypeptide
METGLRWLLLVAVLKGVQCQSLEESGGDLVKPGASLTLTCKASGFDFSSSYYMCWVRQAPGRLEWIACIYGGGLSNTYYAGWAKGRFTISKTSSTTVTLQMTSLTVADTATYFCARDAGTSGDYLNLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELPGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPTVLDSDGSYFLYSKLSVPTSEWQRGDVFTCSVMHEALHNHYTQKSISRSPGK

SEQ ID NO: 65 - protein, 462 aa, synthetic construct
  REGION 1..462: note = Description of Artificial Sequence: Synthetic polypeptide
METGLRWLLLVAVLKGVQCQQQLEESGGGLVQPEGSLTLTCIASGFSFSSSHWICWVRQAPGKGLEWIACMSTSSGSTYDANWAKGRFTISKTSSTTVTLQMTSLTAADTATYFCARDVGGSTTYFDLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELPGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPTVLDSDGSYFLYSKLSVPTSEWQRGDVFTCSVMHEALHNHYTQKSISRSPGK

SEQ ID NO: 66 - protein, 462 aa, synthetic construct
  REGION 1..462: note = Description of Artificial Sequence: Synthetic polypeptide
METGLRWLLLVAVLKGVQCQSLEESGGDLVKPGASLTLTCTASGFSFSSSYYMCWVRQAPGKRLEWIACIYGGGLSNTYYAGWAKGRFTISKTSSTTVTLQMTSLTAADTATYFCARDAGTSGDYLNLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELPGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPTVLDSDGSYFLYSKLSVPTSEWQRGDVFTCSVMHEALHNHYTQKSISRSPGK

SEQ ID NO: 67 - protein, 465 aa, synthetic construct
  REGION 1..465: note = Description of Artificial Sequence: Synthetic polypeptide
METGLRWLLLVAVLKGVQCQQQLEESGGGLVKPGGTLTLTCKASGIDFSNYYYMCWVRQAPGKGLELIACIYTGSSGSTWYATWAKGRFTISKTSSTTVTLQMTSLTAADTATYFCARDRDVGSLYDSLDLWGQGTLVTVSPGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELPGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPTVLDSDGSYFLYSKLSVPTSEWQRGDVFTCSVMHEALHNHYTQKSISRSPGK

SEQ ID NO: 68 - protein, 463 aa, synthetic construct
  REGION 1..463: note = Description of Artificial Sequence: Synthetic polypeptide
METGLRWLLLVAVLKGVQCQEQLVESGGGLVQPEGSLTLTCTASGFSFSDSYYMCWVRQAPGKGLEWIACIYGGTITNTYYASWAKGRFTISKTSSTTVTLQMTSLTAADTATYFCARDLGAAGDAYNLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELPGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPTVLDSDGSYFLYSKLSVPTSEWQRGDVFTCSVMHEALHNHYTQKSISRSPGK

SEQ ID NO: 69 - protein, 236 aa, synthetic construct
  REGION 1..236: note = Description of Artificial Sequence: Synthetic polypeptide
MDTRAPTQLLGLLLLWLPGARCASDMTQTPASVSAAVGGTVTIKCQASESIYSGLAWYQQKPGQPPKLLIFDASDLASGVPSRFKGSRSETEYTLTISDLECADAATYYCQCTDRNSITSYAFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 70 - protein, 238 aa, synthetic construct
  REGION 1..238: note = Description of Artificial Sequence: Synthetic polypeptide
MDTRAPTQLLGLLLLWLPGARCAVVMTQTASPVSGAVGGTVTINCQASQSISSSYLSWYQQKPGQPPKLLIYGASTLASGVPSRFKGSGSGTQFTLTISGVQCDDAATYYCAYDAYRLSSPDNIFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 71 - protein, 236 aa, synthetic construct
  REGION 1..236: note = Description of Artificial Sequence: Synthetic polypeptide
MDTRAPTQLLGLLLLWLPGARCAYDMTQTPASVSEPVGGTVTIKCQASQSISNELSWYQQKPGQPPKLLIYRASTLASGVPSRFKGSGSGTQFTLTINGVECADAATYYCQQGYSISNVDNTFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 72 - protein, 238 aa, synthetic construct
  REGION 1..238: note = Description of Artificial Sequence: Synthetic polypeptide
MDTRAPTQLLGLLLLWLPGARCAVVMTQTASPVSGAVGGTVTINCQASQSISASALSWYQQKPGQPPKLLIYAASTLESGVPSRFKGSGSGTQFTLTISGVQCDDAATYYCAYDGYRLSSADNIFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 73 - protein, 236 aa, synthetic construct
  REGION 1..236: note = Description of Artificial Sequence: Synthetic polypeptide
MDTRAPTQLLGLLLLWLPGARCAYDMTQTPASVEAAVGGTVTINCQASQSINNWLSWYQQKPGQRPKLLIYQASTLASGVSSRFKGSGSGTHFTLTISDLECADAATYYCQQGWSIDDIDNAFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 74 - protein, 236 aa, synthetic construct
  REGION 1..236: note = Description of Artificial Sequence: Synthetic polypeptide
MDTRAPTQLLGLLLLWLPGARCASDMTQTPASVSEPVGGTVTIKCQASENIYSSLAWYQQKPGQPPKLLIYDASNLASGVPSRFSGSGSGTEFTLTISDLECADAATYYCQCTYRSSSSSYAFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC